(19) United States
(12) Patent Application Publication
(10) Pub. No.: US 2018/0016632 A1
(43) Pub. Date: Jan. 18, 2018

(54) SYSTEM AND METHOD FOR TRANSPOSASE-MEDIATED AMPLICON SEQUENCING

(71) Applicant: Kapa Biosystems, Inc., Wilmington, MA (US)

(72) Inventors: Jo-Anne PENKLER, Fish Hoek (ZA); Bronwen MILLER, Cape Town (ZA); Martin RANIK, Cape Town (ZA); Eric van der WALT, Cape Town (ZA)

(21) Appl. No.: 15/648,244

(22) Filed: Jul. 12, 2017

Related U.S. Application Data
(60) Provisional application No. 62/361,347, filed on Jul. 12, 2016.
(60) Provisional application No. 62/402,523, filed on Sep. 30, 2016.

(51) Int. Cl. C12Q 1/68; C12N 15/10

(52) U.S. Cl. CPC C12Q 1/6874; C12Q 1/6855; C12N 15/1065; C12N 15/1082; C12Q 1/6806

(57) ABSTRACT

The present invention provides a method for targeted enrichment of nucleic acids including contacting a nucleic acid including at least one region of interest with a plurality of transposase complexes. Each of the transposase complexes includes at least a transposase and a first polynucleotide having a transposon end sequence and a first label sequence. The method further includes incubating the nucleic acid and the transposase complexes under conditions whereby the nucleic acid is fragmented into a plurality of nucleic acid fragments including first polynucleotide attached to each 5′ end of the nucleic acid fragments. The method further includes selectively amplifying the nucleic acid fragments, thereby enriching for a portion of the nucleic acid fragments including the at least one region of interest relative to a remaining portion of the nucleic acid fragments, and sequencing the enriched nucleic acid fragments.

### CROSS-REFERENCE TO RELATED APPLICATIONS

This application claims priority to U.S. Patent Application No. 62/361,347, filed Jul. 12, 2016 and to U.S. Patent Application No. 62/402,523, filed Sep. 30, 2016, the contents of each of which are herein incorporated by reference in their entirety.

### INCORPORATION OF SEQUENCE LISTING

The contents of the text file named “RMSI-011-001US_SL.txt”, which was created on Jul. 12, 2017 and is 36,453 bytes in size, are hereby incorporated by reference in their entirety.

### BACKGROUND OF THE DISCLOSURE

The disclosure relates, in general, to targeted enrichment of nucleic acids and, more particularly, to a system and method for transposase-mediated fragmentation and amplification-based enrichment with unidirectional sequence-specific primers.

Whole genome sequencing is a valuable tool for both research and clinical applications. For example, sequencing can provide a comprehensive view of the entire genome and allow for the detection of single nucleotide variants, nucleotide insertions and deletions, and large structural variants. However, sequencing entire genomes can be costly, and researchers and clinicians may only be interested in genetic information from particular regions of interest. In these cases, target enrichment ahead of sequencing is a more attractive option.

Targeted enrichment methods for sequencing can be broadly divided into two categories: i) hybridization-based capture methods, and ii) polymerase chain reaction (PCR)-based (i.e., amplification-based) enrichment methods. For many applications, hybridization methods can be more sensitive methods for identifying single nucleotide polymorphisms (SNPs) at low minor allele frequencies, but may suffer from a need for large starting material input requirements (e.g., more than 100 ng of DNA or RNA), laborious workflows (e.g., time intensive, extensive hands-on time), and high costs. PCR methods are generally less sensitive with respect to SNP detection as the priming sites are always the same for each of the different templates in a sample, which means that PCR duplicates cannot be identified. Novel gene fusion events can also not be identified by standard amplification-based methods, because only targets containing primer binding sites for both forward and reverse primers are amplified and subsequently sequenced.

Accordingly, there is a need for improved processes and systems for targeted enrichment for sequencing.

### SUMMARY OF THE DISCLOSURE

The present invention overcomes the aforementioned drawbacks by providing a system and method for transposase-mediated amplification-based sequencing.

In accordance with one aspect of the present disclosure, method for targeted enrichment of nucleic acids includes contacting a nucleic acid including at least one region of interest with a plurality of transposase complexes. Each of the transposase complexes includes at least a transposase and a first polynucleotide having a transposon end sequence and a first label sequence. The method further includes incubating the nucleic acid and the transposase complexes under conditions whereby the nucleic acid is fragmented into a plurality of nucleic acid fragments including first polynucleotide attached to each 5′ end of the nucleic acid fragments. The method further includes selectively amplifying the nucleic acid fragments, thereby enriching for a portion of the nucleic acid fragments including the at least one region of interest relative to a remaining portion of the nucleic acid fragments, and sequencing the enriched nucleic acid fragments. In certain embodiments of the methods of the disclosure, the amplifying step comprises a denaturation step. In certain embodiments of the methods of the disclosure, the amplifying step does not comprise a denaturation step and the amplifying step comprises a nick-translation step. By inclusion of the nick-translation step, the amplifying step produces a plurality of nucleic acid fragments including the first polynucleotide attached to each 5′ end of the nucleic acid fragments and the complement of the first polynucleotide attached to each 3′ end of the nucleic acid fragments.

The foregoing and other aspects and advantages of the invention will appear from the following description. In the description, reference is made to the accompanying drawings which form a part hereof, and in which there is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF THE DRAWINGS

FIG. 1A is a schematic illustration showing a first phase in a method for transposase-mediated amplicon sequencing according to the present disclosure. In the first phase of the method, a transposase complex is formed using common end sequences, and the complex is exposed to a nucleic acid sample. Following transposase-based fragmentation and labeling of the nucleic acid sample, the fragmented nucleic acids are denatured in preparation for a second phase of the method.

FIG. 1B is a schematic illustration showing a second phase in the method of FIG. 1A. In the second phase of the method, the fragmented and denatured nucleic acids are amplified using sequence specific primers paired with adapter specific primers for amplification-based enrichment of one or more regions of interest. Dotted lines extending from primers indicate extension of the primer and replication of the template strand by a polymerase.

FIG. 1C is a schematic illustration showing a third phase of the method of FIGS. 1A and 1B. In the third phase of the method, a second primer set is used to further amplify and tag the amplicons generated in the second phase of the method in preparation for sequencing. Dotted lines extending from primers indicate extension of the primer and replication of the template strand by a polymerase.

FIG. 1D is a schematic illustration showing an alternative embodiment of a second phase in the method of FIG. 1A. In the second phase of the method, the fragmented and denatured nucleic acids are amplified using out-nested sequence specific primers paired with adapter specific primers for amplification-based enrichment of one or more regions of interest. Dotted lines extending from primers indicate extension of the primer and replication of the template strand by a polymerase.

FIG. 1E is a schematic illustration showing a third phase of the method of FIGS. 1A and 1D, including a second round of amplification with in-nested target specific primers. In the third phase of the method, a second primer set is used to further amplify and tag the amplicons generated in the second phase of the method in preparation for sequencing. Dotted lines extending from primers indicate extension of the primer and replication of the template strand by a polymerase.

FIG. 2A is schematic illustration of an example method for targeted enrichment of ribonucleic acids (i.e., RNA) including cDNA synthesis, end repair and dA-tailing, adapter ligation, and PCR.

FIG. 2B is a schematic illustration of a method according to the present disclosure for targeted enrichment of RNA including transposase based fragmentation and labeling of nucleic acids, followed by PCR. Notably, the method of FIG. 2B eliminates a number of steps present in the method of FIG. 2A.

FIG. 3A is a schematic illustration of another example method for targeted enrichment of deoxyribonucleic acids (i.e., DNA) including fragmentation, end repair and dA-tailing, adapter ligation, and PCR.

FIG. 3B is a schematic illustration of a method according to the present disclosure for targeted enrichment of DNA including transposase based fragmentation and labeling of nucleic acids, followed by PCR. Notably, the method of FIG. 3B eliminates a number of steps present in the method of FIG. 3A.

FIG. 4 is a plot of real-time PCR data showing normalized fluorescence signal as a function of cycle number for data collected according to an embodiment of the system and method of the present disclosure. Real-time PCR data was collected for four different samples (curves labeled A-D): Curve A—positive control (unsheared DNA pre-amplified with gene specific primers); Curve B—modified TnPrep library (TnPrep with gene specific primers for targeted amplification using a 2-step PCR protocol); Curve C—standard TnPrep library (TnPrep with non-specific amplification); Curve D—negative control (unsheared DNA). Data generated using primers specific to regions of interest showed approximately 1000-fold enrichment for region of interest in the modified TnPrep library (curve B) compared to the standard TnPrep library (curve C). The “standard TnPrep library” is a library prepared with the standard transposase loaded with R1 and R2 arms and amplified with the standard NEXTERA i5 and i7 primers (ILLUMINA, INC.), as opposed to the “modified TnPrep library” which is prepared with a transposase loaded with only R1 arms and amplified with NEXTERA i5 and target specific primers.

FIG. 5 is a nucleic acid analysis trace showing fluorescence signal as a function of nucleic acid size (bp) for sequencing libraries prepared according to the methods of the present disclosure (including a 2-step PCR protocol), and with higher concentrations of transposase (36 μg/ml). Data is shown for i) sequencing libraries prepared with sequence specific primers to enrich for target regions of interest, and ii) standard sequencing libraries prepared without specific primers.

FIG. 6 is a nucleic acid analysis trace showing fluorescence signal as a function of nucleic acid size (bp) for sequencing libraries prepared according to the methods of the present disclosure (using a 2-step PCR protocol), and with lower concentrations of transposase (4 μg/ml). Data is shown for i) sequencing libraries prepared with sequence specific primers to enrich for target regions of interest, and ii) standard sequencing libraries prepared with non-specific primers.

FIG. 7 is a bar chart illustrating the specificity of amplification assessed by real-time PCR using primers specific to the adapter sequences, and in separate reactions, primers specific to the target regions. In the situation where all fragments with adapter sequences on both ends contain the region of interest, the difference between the two Cₜ (i.e., the threshold cycle) values is expected to be minimal. For non-specific amplification, fragments can result that have both adapter sequences, but do not contain the region of interest, and in this case the difference between the Cₜ values would be larger. The bar chart illustrates that for both the 2-step and 3-step amplification/PCR workflows/protocols the delta Cₜ is low, indicating specific amplification of regions of interest. Labels on the horizontal axis indicate results for libraries prepared with different cycle numbers as follows (notation is in the format: [sample #]-[# of cycles in 1^{st} round of PCR]/[# of cycles in 2^{nd} round of PCR]/[# of cycles in 3^{rd} round of PCR (if applicable)]): 7-20/12; 8-15/15; 9-10/20; 10-20/8/8; 11-15/10/7; 12-10/10/12.

FIG. 8 is a bar chart illustrating percent on target rate as determined by sequencing. Highly specific amplification and successful enrichment of regions of interest, with greater than 90% on target rate was achieved in all cases. Labels on the horizontal axis indicate results for libraries prepared with different cycle numbers as follows (notation is in the format: [sample #]-[# of cycles in 1^{st} round of PCR]/[# of cycles in 2^{nd} round of PCR]/[# of cycles in 3^{rd} round of PCR (if applicable)]): 7-20/12; 8-15/15; 9-10/20; 10-20/8/8; 11-15/10/7; 12-10/10/12.

FIG. 9 is a bar graph showing the effect of transposase complex concentration on the percentage of on-target reads.

FIG. 10A is a bar graph showing the effect of DNA input on the percentage of on-target reads.

FIG. 10B is plot of normalized coverage as a function of target DNA amplicon showing the effect of DNA input on coverage uniformity.

FIG. 11 is a parity plot comparing observed and expected alternate allele frequencies for a library prepared from 50 ng of a reference standard including known variants. Error bars are indicative of the standard deviation from 3 technical replicates.

FIG. 12 is a bar graph illustrating the on-target rates of libraries constructed from different FFPET DNA samples according to the present disclosure.

FIG. 13 is a bar graph illustrating the on-target rates of libraries constructed from reactions wherein the heat denaturation step is eliminated and wherein a nick-translation step has been added. The results of the nick-translation step were compared to a heat-treated condition. The nick-translation step was included to ensure that an adapter would be present on both 5′ and 3′ ends.

FIG. 14A is a bar graph illustrating quality control of fragments prior to sequencing. Similar to FIG. 13, some reactions included a heat treatment step (HT) while others included a nick-translation step (NT). The R1 arms used in these reactions include molecular barcodes (also referred to herein as unique molecular identifiers (UIDs)) in place of i5 index (See Example 6).

FIG. 14B is a series of graphs illustrating the use of standard versus UID-containing R1 arms in either a 31-primer or a 40-primer panel.

The Like numbers will be used to describe like parts from Figure to Figure throughout the following detailed description.

### DETAILED DESCRIPTION OF THE DISCLOSURE

### 1. Definitions

The terms “a”, “an” and “the” generally include plural referents, unless the context clearly indicates otherwise.

The term “amplification” generally refers to the production of a plurality of nucleic acid molecules from a target nucleic acid wherein primers hybridize to specific sites on the target nucleic acid molecules in order to provide an initiation site for extension by a polymerase. Amplification can be carried out by any method generally known in the art, such as but not limited to: standard PCR, long PCR, hot start PCR, qPCR, RT-PCR and Isothermal Amplification. Other amplification reactions comprise, among others, the Ligase Chain Reaction, Polymerase Ligase Chain Reaction, Gap-LCR, Repair Chain Reaction, 3SR, NASBA, Strand Displacement Amplification (SDA), Transcription Mediated Amplification (TMA), and Qb-amplification.

The term “complementary” generally refers to the ability to form favorable thermodynamic stability and specific pairing between the bases of two nucleotides at an appropriate temperature and ionic buffer conditions. This pairing is dependent on the hydrogen bonding properties of each nucleotide. The most fundamental examples of this are the hydrogen bond pairs between thymine/adenine and cytosine/guanine bases. In the present invention, primers for amplification of target nucleic acids can be both fully complementary over their entire length with a target nucleic acid molecule and “semi-complementary” wherein the primer contains additional, non-complementary sequence minimally capable or incapable of hybridization to the target nucleic acid.

The term “detecting” as used herein relates to a qualitative test aimed at assessing the presence or absence of a target nucleic acid in a sample.

The term “enriched” as used herein relates to any method of treating a sample comprising a target nucleic acid that allows for separating the target nucleic acid from at least a part of other material present in the sample. “Enrichment” can, thus, be understood as a production of a higher amount of target nucleic acid over other material.

The term “excess” generally refers to a larger quantity or concentration of a certain reagent or reagents as compared to another.

The term “hybridize” generally refers to the base-pairing between different nucleic acid molecules consistent with their nucleotide sequences. The terms “hybridize” and “anneal” can be used interchangeably.

The terms “nucleic acid” or “polynucleotide” can be used interchangeably and refer to a polymer that can be corresponded to a ribose nucleic acid (RNA) or deoxyribose nucleic acid (DNA) polymer, or an analog thereof. This includes polymers of nucleotides such as RNA and DNA, as well as synthetic forms, modified (e.g., chemically or biochemically modified) forms thereof, and mixed polymers (e.g., including both RNA and DNA subunits). Exemplary modifications include methylation, substitution of one or more of the naturally occurring nucleotides with an analog, internucleotide modifications such as uncharged linkages (e.g., methyl phosphonates, phosphotriesters, phosphoamidates, carbamates, and the like), pendent moieties (e.g., polypeptides), intercalators (e.g., acridine, psoralen, and the like), chelators, alkylators, and modified linkages (e.g., alpha anomeric nucleic acids and the like). Also included are synthetic molecules that mimic polynucleotides in their ability to bind to a designated sequence via hydrogen bonding and other chemical interactions. Typically, the nucleotide monomers are linked via phosphodiester bonds, although synthetic forms of nucleic acids can comprise other linkages (e.g., peptide nucleic acids as described in Nielsen et al. (Science 254:1497-1500, 1991). A nucleic acid can be or can include, e.g., a chromosome or chromosomal segment, a vector (e.g., an expression vector), an expression cassette, a naked DNA or RNA polymer, the product of a polymerase chain reaction (PCR), an oligonucleotide, a probe, and a primer. A nucleic acid can be, e.g., single-stranded, double-stranded, or triple-stranded and is not limited to any particular length. Unless otherwise indicated, a particular nucleic acid sequence comprises or encodes complementary sequences, in addition to any sequence explicitly indicated.

The term “nucleotide” in addition to referring to the naturally occurring ribonucleotide or deoxyribonucleotide monomers, shall herein be understood to refer to related structural variants thereof, including derivatives and analogs, that are functionally equivalent with respect to the particular context in which the nucleotide is being used (e.g., hybridization to a complementary base), unless the context clearly indicates otherwise.

The term “oligonucleotide” refers to a nucleic acid that includes at least two nucleic acid monomer units (e.g., nucleotides). An oligonucleotide typically includes from about six to about 175 nucleic acid monomer units, more typically from about eight to about 100 nucleic acid monomer units, and still more typically from about 10 to about 50 nucleic acid monomer units (e.g., about 15, about 20, about 25, about 30, about 35, or more nucleic acid monomer units). The exact size of an oligonucleotide will depend on many factors, including the ultimate function or use of the oligonucleotide. Oligonucleotides are optionally prepared by any suitable method, including, but not limited to, isolation of an existing or natural sequence, DNA replication or amplification, reverse transcription, cloning and restriction digestion of appropriate sequences, or direct chemical synthesis by a method such as the phosphotriester method of Narang et al. (Meth. Enzymol. 68:90-99, 1979); the phosphodiester method of Brown et al. (Meth. Enzymol. 68:109-151, 1979); the diethylphosphoramidite method of Beaucage et al. (Tetrahedron Lett. 22:1859-1862, 1981); the triester method of Matteucci et al. (J. Am. Chem. Soc. 103:3185-3191, 1981); automated synthesis methods; Maskless Array Synthesis as disclosed in Singh-Gasson et al., Nature Biotechnology, 17: 974-978, 1999, or the solid support method of U.S. Pat. No. 4,458,066, or other methods known to those skilled in the art.

The term “primer” refers to a polynucleotide capable of acting as a point of initiation of template-directed nucleic acid synthesis when placed under conditions in which polynucleotide extension is initiated (e.g., under conditions comprising the presence of requisite nucleoside triphosphates (as dictated by the template that is copied) and a polymerase in an appropriate buffer and at a suitable temperature or cycle(s) of temperatures (e.g., as in a polymerase chain reaction)). To further illustrate, primers can also be used in a variety of other oligonucleotide-mediated synthesis processes, including as initiators of de novo RNA synthesis and in vitro transcription-related processes (e.g., nucleic acid sequence-based amplification (NASBA), transcription mediated amplification (TMA), etc.). A primer is typically a single-stranded oligonucleotide (e.g., oligodeoxyribonucleotide). The appropriate length of a primer depends on the intended use of the primer but typically ranges from 6 to 40 nucleotides, more typically from 15 to 35 nucleotides. Short primer molecules generally require cooler temperatures to form sufficiently stable hybrid complexes with the template. A primer need not reflect the exact sequence of the template but must be sufficiently complementary to hybridize with a template for primer elongation to occur. In certain embodiments, the term “primer pair” means a set of primers including a 5′ sense primer (sometimes called “forward”) that hybridizes with the complement of the 5′ end of the nucleic acid sequence to be amplified and a 3′ antisense primer (sometimes called “reverse”) that hybridizes with the 3′ end of the sequence to be amplified (e.g., if the target sequence is expressed as RNA or is an RNA). A primer can be labeled, if desired, by incorporating a label detectable by spectroscopic, photochemical, biochemical, immunochemical, or chemical means. For example, useful labels include 32P, fluorescent dyes, electron-dense reagents, enzymes (as commonly used in ELISA assays), biotin, or haptens and proteins for which antisera or monoclonal antibodies are available.

In the sense of the invention, “purification”, “isolation” or “extraction” of nucleic acids relate to the following: Before nucleic acids may be analyzed in a diagnostic assay e.g. by amplification, they typically have to be purified, isolated or extracted from biological samples containing complex mixtures of different components. For the first steps, processes may be used which allow the enrichment of the nucleic acids. Such methods of enrichment are described herein.

The term “quantitating” as used herein relates to the determination of the amount or concentration of a target nucleic acid present in a sample.

“Target nucleic acid” is used herein to denote a nucleic acid in a sample which should be analyzed, i.e. the presence, non-presence, nucleic acid sequence and/or amount thereof in a sample should be determined. The target nucleic acid may be a genomic sequence, e.g. part of a specific gene, RNA, cDNA or any other form of nucleic acid sequence. In some embodiments, the target nucleic acid may be viral or microbial.

The terms “target nucleic acid”, and “target molecule” can be used interchangeably and refer to a nucleic acid molecule that is the subject of an amplification reaction that may optionally be interrogated by a sequencing reaction in order to derive its sequence information.

The terms “target specific region” or “region of interest” can be used interchangeably and refer to the region of a particular nucleic acid molecule that is of scientific interest. These regions typically have at least partially known sequences in order to design primers which flank the region or regions of interest for use in amplification reactions and thereby recover target nucleic acid amplicons containing these regions of interest.

The term “maskless array synthesis” (MAS) refers to light-directed synthesis of oligonucleotides on the surface of a substrate as an array in the absence of a physical mask, such as the method as described by Singh-Gasson et al., Nature Biotech, 17: 974-978 (October 1999), the teachings of which are hereby incorporated by reference. Briefly, the MAS technique generally uses a digital microarray mirror device (DMD) which consists of micromirrors to form virtual masks. These mirrors are individually addressable and can be used to create any given pattern or image in a broad range of wavelengths. The DMD forms an image on the surface of the substrate, wherein the substrate contains chemical moieties that are activated by light. A solution containing a given nucleotide is then washed over the surface of the substrate, and binds to the activated regions. The nucleotide in the solution contains are photoprotected with a protecting group that is photolabile. In a second round of synthesis, the DMD forms a second image onto selected regions of the substrate, thereby selectively activating the substrate in those regions, and a second given nucleotide (again, photoprotected) is washed over the substrate. This second nucleotide binds to those regions that have been activated during the second round of illumination. Thus, selected nucleotides can be added to selected regions, allowing for synthesis of an array of oligonucleotides through light-directed synthesis in the absence of a mask. This process is repeated numerous times in order to build the oligonucleotides sequences on a monomer-by-monomer basis.

Other methods of building arrays can also be used in the present invention, such as the use of chromium masks or spotting of oligonucleotides on an array. MAS provides improved flexibility and simplicity when used in the present invention, but other means of forming arrays are useful as well. Examples of the synthetic systems, besides MAS, that can be used in the present invention are those well-known methods used by Affymetrix, Oxford Gene Technologies, and Agilent.

### 2. Description

The first clinically relevant gene fusion (BCR-Abl) was identified in 1960, and is formed as a result of a translocation between chromosomes 9 and 22. The resulting fusion protein, an unregulated mutant tyrosine kinase, leads to uncontrolled cell growth and the development of the blood cancer, chronic myelogenous leukemia. Based on an understanding of the fusion product, a targeted therapy could be developed that makes use of tyrosine kinase inhibitors. In recent years, fusion genes have also been identified in numerous solid tumor cancers including colorectal, lung, prostate, breast and stomach. The abnormal protein products of fusion genes are active only in the cancer cells, and are thus potentially good targets for drug intervention with minimal toxic side effects.

Existing target enrichment methods capable of detecting novel gene fusions include the Archer Fusion Plex kit (Archer Dx) which requires cDNA synthesis followed by end repair, dA-tailing and adapter ligation followed by two PCR steps. Similarly, NuGEN's Single Primer Enrichment Technology also requires separate steps for fragmentation, end repair and ligation of adapters, prior to annealing and extension of target specific probes and a subsequent PCR step.

In general, the present disclosure provides for a combination of “transposase-based library preparation” (hereinafter, “TnPrep”), and multiplexed amplicon sequencing. In one embodiment the present disclosure provides for a method combining a modified TnPrep approach with one or more primers targeting a specific region of interest for targeted sequencing. In one aspect, embodiments of TnPrep can employ a transposition reaction to simultaneously fragment and tag DNA. Examples of tags that can be appended during the fragmentation process include nucleic acid sequence tags for a given sequencing platform, unique barcodes sequences, sample index sequences, the like, and combinations thereof.

The proposed invention has a simple and relatively fast workflow including fragmentation and simultaneous addition of sequencing adapters to the 5′-ends of the fragmented target DNA (TnPrep) by incubating target DNA with a transposase enzyme containing arms with a portion of the adapter sequences required for platform specific sequencing (e.g., ILLUMINA sequencing). The reaction product can be treated to ensure that no nick translation takes place in the subsequent amplification steps, and thus fragments only contain a single adapter sequence at the 5′-ends. PCR amplification can be performed with standard indexing primers to complete the adapter sequence and add any necessary indices, as well as a primer (or primers) targeting the region(s) of interest (ROI). In one example, a first round of PCR with target-specific primers is performed, followed by a second PCR with in-nested target specific primers that can include an out-nested portion containing part of the adapter sequence. Thereafter, a final PCR step adds in the sequencing index, producing libraries that are ready for sequencing. It is also possible to combine the second and final PCR steps into one reaction.

Notably, the present disclosure demonstrated a number of advantages over alternative approaches to targeted enrichment for library preparation. In one aspect, the low start site complexity and inability to identify duplicates inherent in traditional PCR-based (i.e., amplicon) sequencing methods is addressed by the present disclosure as TnPrep generates a diversity of start sites and offers the opportunity to introduce unique molecular identifiers (UIDs) to each nucleic acid fragment. In another aspect, embodiments of the present disclosure provides for the identification of novel gene fusion events, as there is only a single target-specific primer. Accordingly, prior knowledge of the fusion partner or breakpoint is not required. In yet another aspect, the workflows described herein are straightforward and relatively fast in comparison with alternative approaches. In yet a further aspect, embodiments of the present disclosure are applicable to a variety of starting materials including both RNA and DNA.

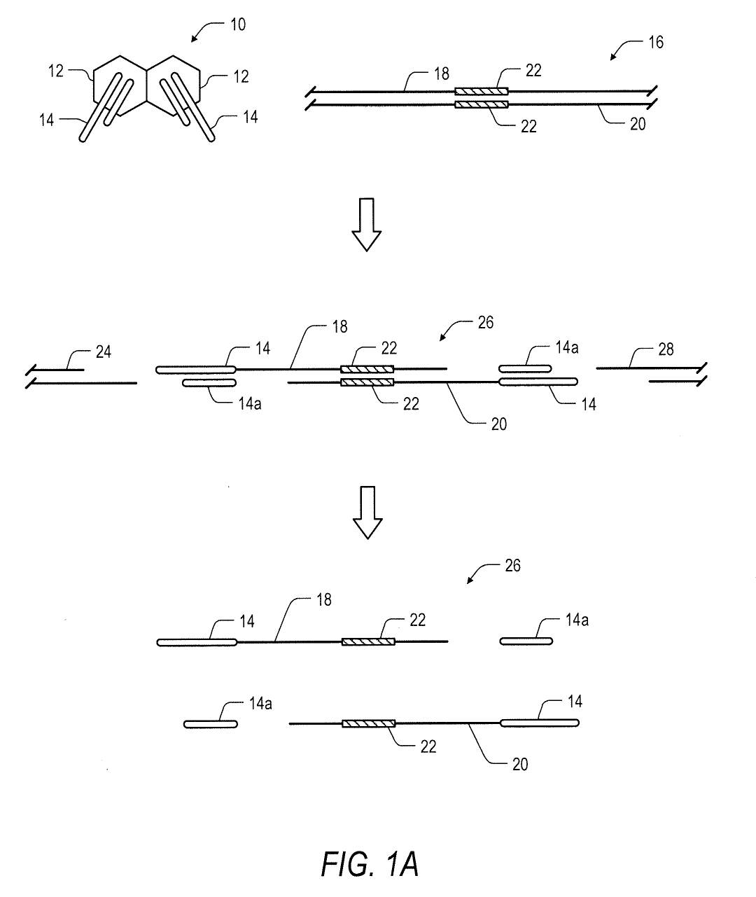

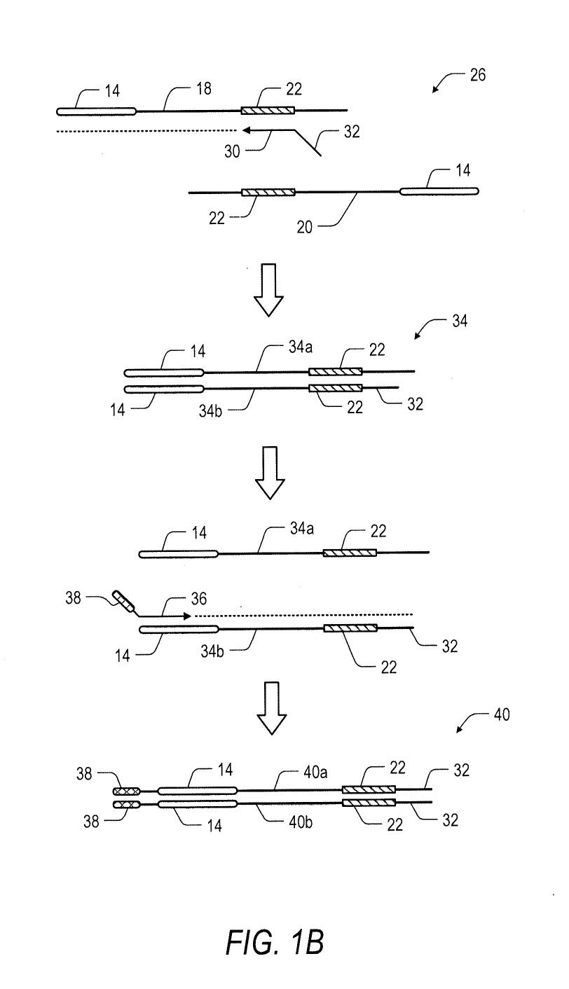

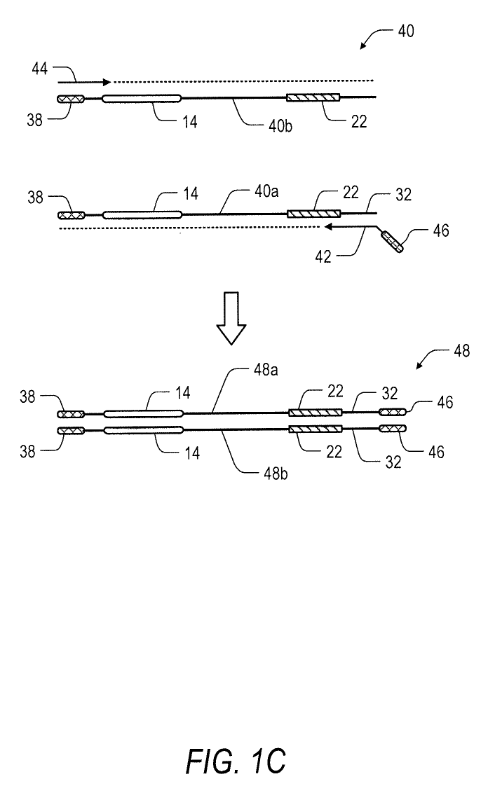

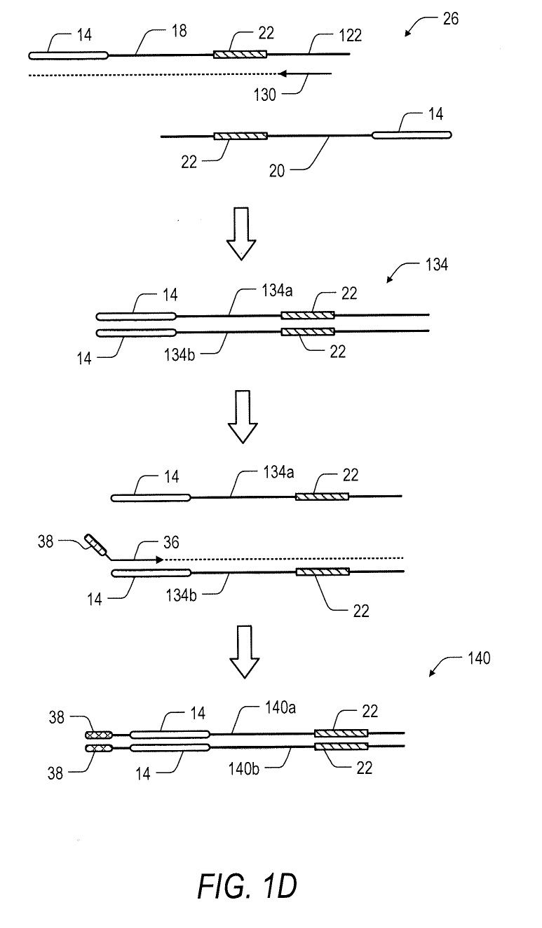

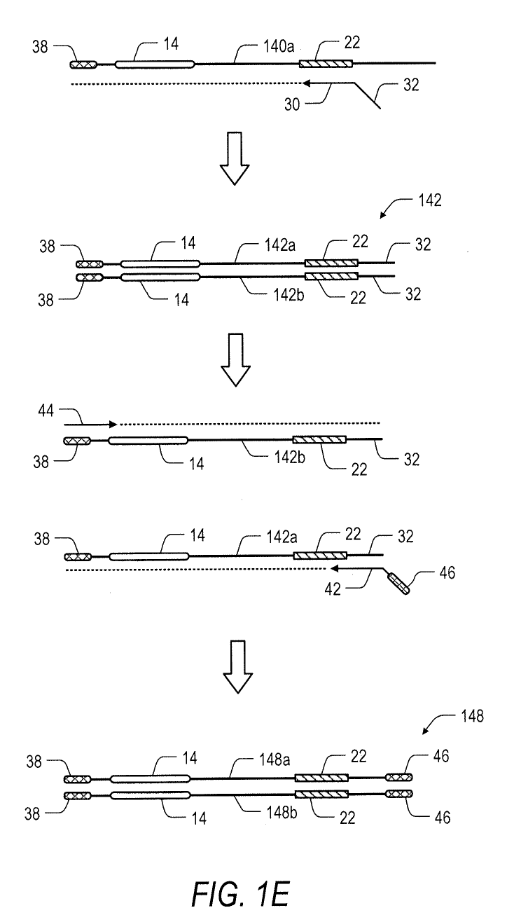

Turning now to FIGS. 1A to 1C a one method of target enrichment including a 2-step PCR workflow for target enrichment includes preparation of a transposase complex 10. The transposase complex includes a pair of transposase enzymes 12, where each of the transposase enzymes includes a first polynucleotide 14 having a transposon end sequence and a label sequence. Notably, the transposon end sequence corresponds to the first transposase. In one aspect, the transposase is a hyperactive transposase such as hyperactive Tn5. In another aspect the transposase is wild-type or mutant transposase derived from another source organism, such as Alishewanella aestuarii, Vibrio cholerae, and Vibrio harveyi. In yet another aspect, another like transposase enzyme may be used.

The transposase complex 10 is incubated with a nucleic acid 16, such as a genomic DNA fragment. The nucleic acid 16 includes a top strand 18 and a bottom strand 20, as well as a target region or region of interest (ROI) 22. Following incubation, the transposase complex 10 concomitantly fragments the nucleic acid 16 into a plurality of fragments including the fragment 24, the fragment 26, and the fragment 28. The fragment 26 includes the ROI 22, and further includes the polynucleotides 14 attached to the 5′ ends of the top strand 18 and bottom strand 20 of the fragment 26.

In one aspect, the fragment 26 can be denatured without the need for a gap fill reaction and ligation or PCR extension step to further prepare the fragments as in other TnPrep methods described elsewhere. Accordingly, when the fragment 26 is denatured, the portions 14a of the polynucleotides 14 can become dissociated from top strand 18 and bottom strand 20.

Turning to FIG. 1B, a first round of PCR amplification can be performed on the top strand 18 and bottom strand 20 of the fragment 26. A region specific primer 30 including a tail sequence 32 is complementary to the ROI 22, and can therefore be used to selectively enrich for the ROI 22. In one aspect, the region specific primer 30 can be used to target only one strand (e.g., top strand 18) of the (denatured) fragment 26.

The amplification product 34 (including top strand 34a and bottom strand 34b) following the first round extension with the primer 32 can be denatured to provide a template strand (i.e., bottom strand 34b) for a label specific primer 36 having a tail sequence 38. Notably, ROI 22 can only be exponentially amplified if the fragment 26 includes the ROI 22 proximal to the polynucleotide 14 to provide the amplification product 40 (including top strand 40a and bottom strand 40b).

Turning to FIG. 1C, The amplification product 40 is further amplified in a second round of PCR using a first primer 42 complementary to at least a portion of the tail sequence 32 added by the region specific primer 30, and second primer 44 complementary to at least a portion of the sequence 38 added by the label specific primer 36. The first primer 42 can also include a tail sequence 46. In one aspect, the tail sequence 38 and the tail sequence 46 can include labels, adapters, or indexes for platform specific sequencing. The resulting enriched amplification product 48 (including top strand 48a and bottom strand 48b) can then be sequenced.

Turning now to FIGS. 1D and 1E, another method of target enrichment according to the present disclosure can include a 3-step PCR workflow. In the 3-step workflow, the fragment 26 is prepared as previously described and illustrated with respect to FIG. 1A. However, in contrast to the 2-step PCR workflow illustrated in FIGS. 1B and 1C, the 3-step PCR workflow includes a first out-nested round of PCR step followed by a second in-nested round of PCR, and a third and final round of PCR to add terminal adapter sequence(s). The second and third rounds of PCR are comparable to the workflow illustrated in FIGS. 1B and 1C. In one aspect, it can be useful to employ a nested PCR strategy to improve specificity and achieve a higher on-target rate. However, in some embodiments, the second and third rounds of PCR can be combined into a single reaction while still preserving the nested PCR approach described herein.

Referring to FIG. 1D, a first (out-nested) round of PCR amplification can be performed on the top strand 18 and bottom strand 20 of the fragment 26. An out-nested region specific primer 130 is complementary to a sequence 122 downstream of ROI 22 and can be used to selectively enrich for the ROI 22. In one aspect, the region specific primer 130 can be used to target only one strand (e.g., top strand 18) of the fragment 26. The amplification product 134 (including top strand 134a and bottom strand 134b) following the first round extension with the region specific primer 130 can be denatured to provide a template strand (i.e., bottom strand 134b) for the label-specific primer 36 having the tail sequence 38. Notably, ROI 22 can only be exponentially amplified if the fragment 26 includes the ROI 22 proximal to the polynucleotide 14 to ultimately provide the amplification product 140 (including top strand 140a and bottom strand 140b).

Referring to FIG. 1E, a second (in-nested) round of PCR amplification including the top strand 140a of the amplification product 140 can be performed with the region specific primer 30 including the tail sequence 32 which is complementary to the ROI 22. In one aspect, exponential amplification can be achieved through the inclusion of a suitable second primer, such as the label-specific primer 36. The in-nested round of PCR selectively enriches for the ROI 22. Following the second round extension with the primer 30, the amplification product 142 (including top strand 142a and bottom strand 142b) can be denatured to provide a template strand (i.e., bottom strand 142a) for the label specific first primer 42. Accordingly, the amplification product 142 is further amplified in a final round of PCR using the first primer 42 complementary to at least a portion of the tail sequence 32 added by the region specific primer 30, and second primer 44 complementary to at least a portion of the sequence 38 added by the label specific primer 36. The first primer 42 can also include a tail sequence 46. In one aspect, the tail sequence 38 and the tail sequence 46 can include labels, adapters, or indexes for platform specific sequencing. The resulting enriched amplification product 148 can then be sequenced. As discussed above, the second (in-nested) round and the final round of PCR illustrated in FIG. 1E can be combined into a single round of PCR. Moreover, while the amplification product 48 and the amplification product 148 can be similar or identical depending on the design of the primers used in the workflow, the nested strategy used to prepare the amplification product 148 can provide for improved specificity and a higher on-target rate.

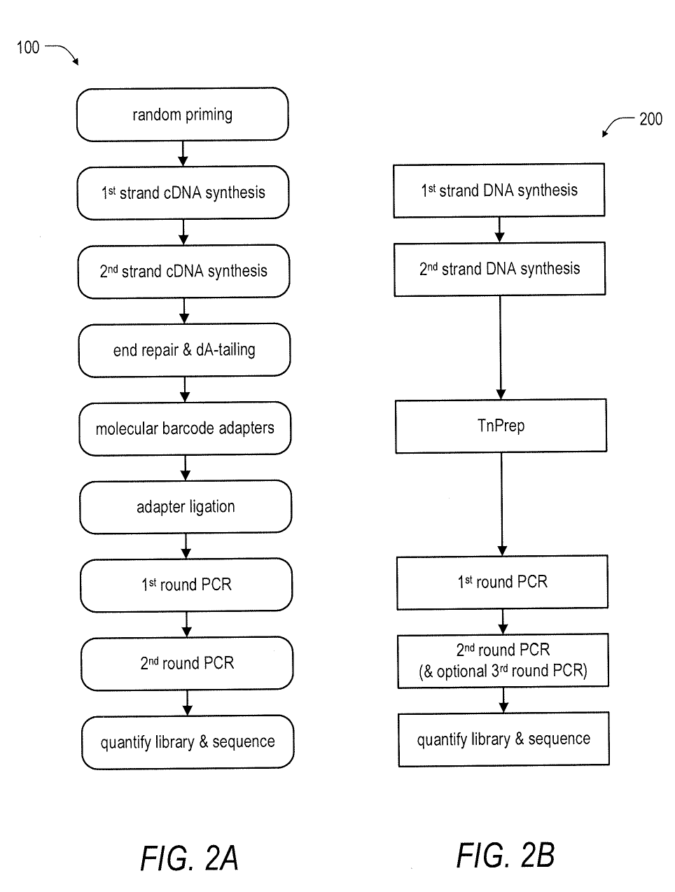

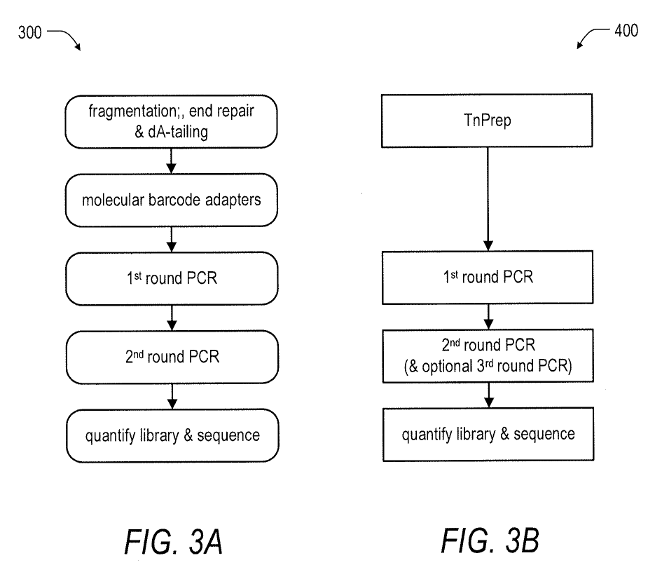

Turning to FIG. 2A, an example of a standard library preparation method 100 is shown. In comparison, FIG. 2B shows a method 200 according to the present disclosure that includes transposase-mediated amplicon sequencing or TnPrep. Notably, the method 200 includes fewer steps overall as compared with the method 100. Moreover, the method 200 excludes several steps included in the method 100, such as end repair, dA-tailing, and adapter ligation. In another example shown in FIGS. 3A and 3B, a method 300 for library preparation is compared with a method 400 for transposase-mediated amplicon sequencing or TnPrep according to the present disclosure. Again, the method 400 includes fewer steps overall as compared with the method 300. Further, the method 400 excludes several steps included in the method 300, such as end repair and dA-tailing.

### EXAMPLES

### Example 1

Libraries were prepared from 50 ng Coriell DNA (NA12878) with a mix of 3 target specific primers. These libraries were sequenced to verify enrichment for targets of interest versus, for example, off-target amplification.

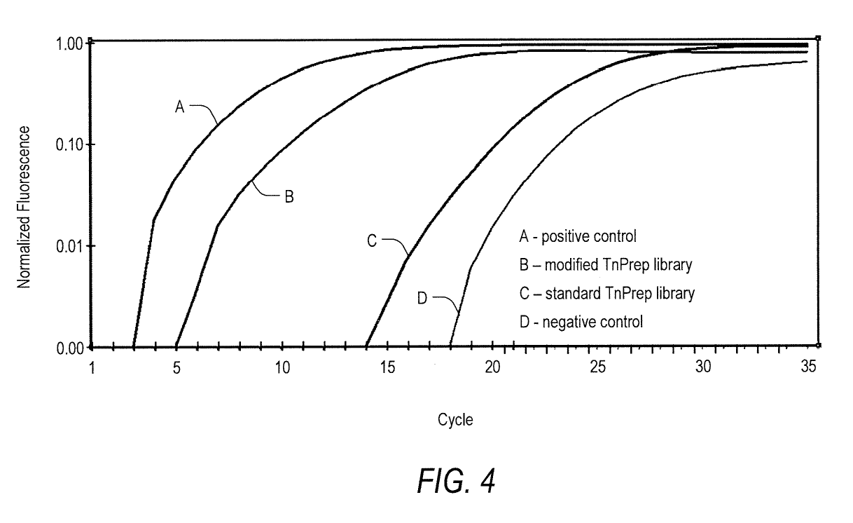

Target specific primers were used to amplify 1 ng of the final library to check that enrichment for the region of interest had taken place by qPCR and end-point PCR. Results from qPCR and Bioanalyzer traces are shown in FIG. 4 and FIGS. 5-6, respectively.

As shown in FIG. 4, real-time amplification using primers specific to region of interest shows approximately 1000-fold enrichment for region of interest in the modified TnPrep library of the present disclosure as compared to the standard TnPrep library. The standard TnPrep library is a library prepared with the standard transposase loaded with R1 and R2 arms and amplified with the standard NEXTERA i5 and i7 primers, as opposed to the modified TnPrep library which is prepared with a transposase loaded with only R1 arms and amplified with NEXTERA i5 and target specific primers.

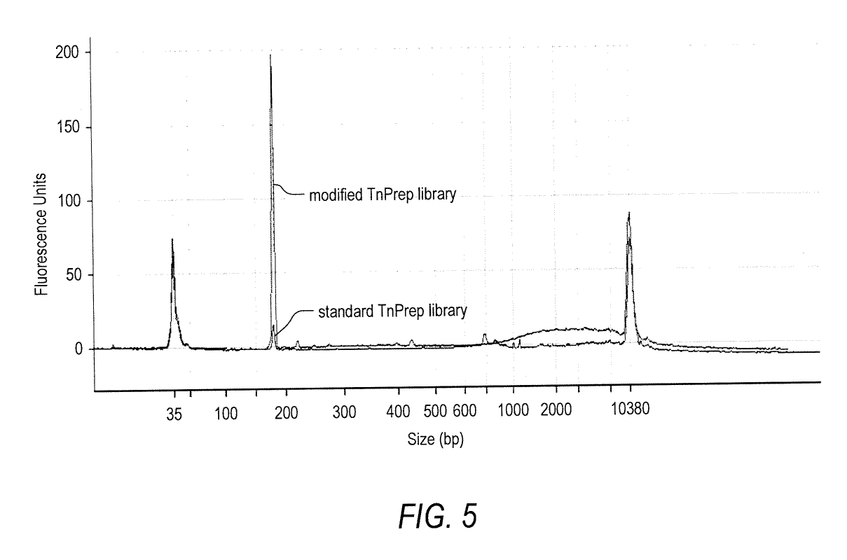

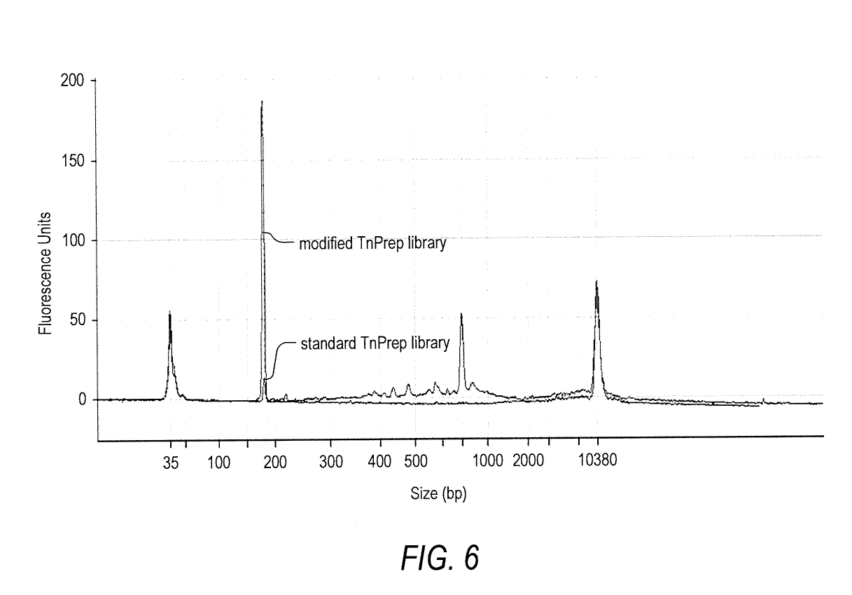

FIGS. 5 and 6 show traces obtained with an AGILENT Bioanalyzer after amplification of libraries with gene specific primers, showing enrichment of region of interest. Traces are from TnPrep libraries prepared with the combination of adapter primer and ROI primer, and from standard TnPrep libraries. Libraries were prepared with a final transposase concentration of either 36 μg/mL (FIG. 5) or 4 μg/mL (FIG. 6).

For the above example and a final transposase concentration of 36 μg/mL, libraries were prepared with pre-loaded transposase containing only R1 arms from 50 ng Coriell DNA (NA12878) as shown in Table 1.

TABLE 1

| Component | Volume (μL) | Final Concentration | |
|---|---|---|---|
| DNA | 4 | 2.5 | ng/μL |
| Reaction buffer | 10 | 1x | |
| MnCl₂ | 2 | 10 | mM |
| Transposase (0.18 mg/ml) | 4 | 36 | μg/mL |

| | | |
|---|---|---|
| Total | 20 | n/a |

Samples were vortexed, spun down and incubated at 55° C. for 5 min.

After incubation, 20 μL GHCl stop solution was added, and the samples were again vortexed, centrifuged and incubated at room temperature for 5 min.

Clean up reactions were carried out with 40 μL SPRI® beads and samples were resuspended to a total volume of 20 μL.

Samples were then heat treated at 95° C. for 5 min to denature strands and prevent nick translation in the subsequent amplification step.

An amplification reaction was set up as shown in Table 2, and samples were cycled in a thermal cycler according the protocol shown in Table 3.

TABLE 2

| Component | Volume (μL) |
|---|---|
| DNA | 10 |
| 5x ReadyMix | 4 |
| i5 primer (0.67 μM) | 3 |
| Reverse primer(s) (0.67 μM) | 3 |
| Total | 20 |

TABLE 3

| Step | Temperature (deg C.) | Time (sec) |
|---|---|---|
| 1 | 99 | 60 |
| 2 | 99 | 15 |
| 3 | 64 | 240 |

Repeat steps 2-3 for a total of 15 cycles

After amplification, samples were cleaned up with 40 μL SPRI beads and resuspended to a final volume of 20 μL. Samples were prepared for a second round of amplification as described in Table 4, and cycled in a thermal cycler according to the protocol shown in Table 5.

TABLE 4

| Component | Volume (μL) |
|---|---|
| DNA | 10 |
| P1 primer (2.5 μM) | 2 |
| Reverse primer (2.5 μM) | 2 |
| Hifi HotStart ReadyMix | 25 |
| Water | 11 |
| Total | 50 |

TABLE 5

| Step | Temperature (deg C.) | Time (sec) |
|---|---|---|
| 1 | 98 | 60 |
| 2 | 98 | 15 |
| 3 | 62 | 30 |
| 4 | 72 | 30 |

Repeat steps 2-4 for a total of 8-10 cycles

| | | |
|---|---|---|
| 5 | 72 | 60 |

Following the second round of amplifications, samples were cleaned up with 50 μL SPRI® beads and resuspended in 20 μL of water. Samples were prepared for a final round of amplification as described in Table 6, and cycled in a thermocycler as described in Table 5.

TABLE 6

| Component | Volume (μL) |
|---|---|
| DNA | 10 |
| P1 primer (2.5 μM) | 5 |
| I7 primer (2.5 μM) | 5 |
| Hifi HotStart ReadyMix | 25 |
| Water | 5 |
| Total | 50 |

Following the final round of amplifications, samples were cleaned up with 50 μL SPRI® beads and resuspended in 20 μL of water.

The oligo sequences used in the above examples are described as follows:

Transposase arms:
Read1 End (R1):
(SEQ ID NO: 1)
TCGTCGGCAGCGTCAGATGTGTATAAGAGACAG.
Amplification primers:
NEXTERA amplification primer (S517):
(SEQ ID NO: 2)
AATGATACGGCGACCACCGAGATCTACACGCGTAAGATCGTCGGCAG
CGTC.

The three outer target specific reverse primers were as follows:

(SEQ ID NO: 3)
CTTCTCCACTTAATAAGCAGTTGAT;
(SEQ ID NO: 4)
TAAGGTATCAATAAATACTCACCAATCTTC;
and
(SEQ ID NO: 5)
CCAGGCTGCCTCACCAT.

The three target specific reverse primers with out-nested portion of adapter sequence were as follows:

(SEQ ID NO: 6)
GTCTCGTGGGCTCGGAGATGTGTATAAGAGACAGTACCAGTGTGTT
ATTCAGGTAGGTCA;
(SEQ ID NO: 7)
GTCTCGTGGGCTCGGAGATGTGTATAAGAGACAGAATTTTCAACTG
CTTTACATAAGAAGCGTT;
and
(SEQ ID NO: 8)
GTCTCGTGGGCTCGGAGATGTGTATAAGAGACAGGTCCTGACTGTG
GCGTCAT.

The sequence of the NEXTERA amplification primer (N70×) was as follows (the I7 representing an index tag):

(SEQ ID NO: 9)
CAAGCAGAAGACGGCATACGAGAT(I7)GTCTCGTGGGCTCGG.
P1 primer:
(SEQ ID NO: 16)
AAT GAT ACG GCG ACC ACC G*A

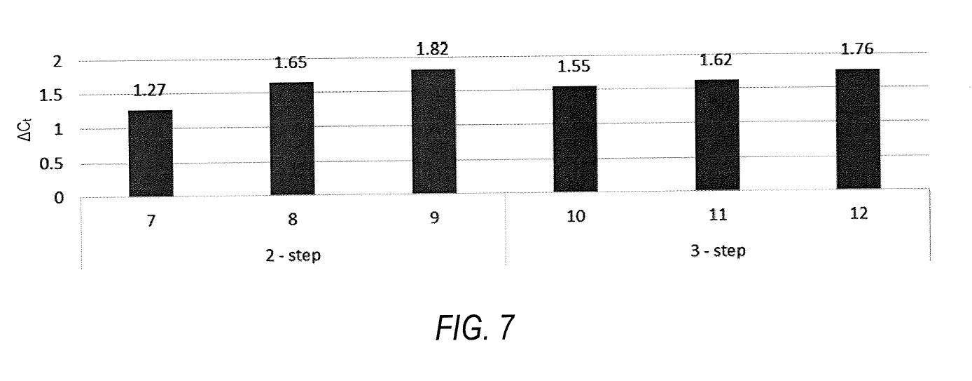

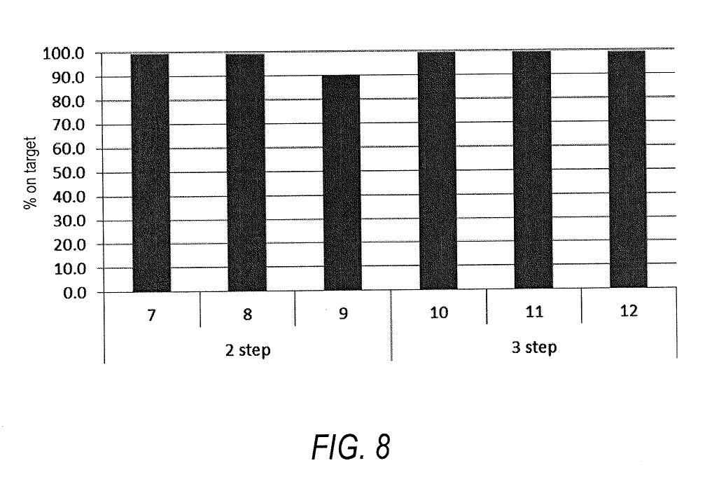

Successful enrichment of targeted regions of interest was confirmed by real time PCR (FIG. 7) and sequencing (FIG. 8).

### Example 2

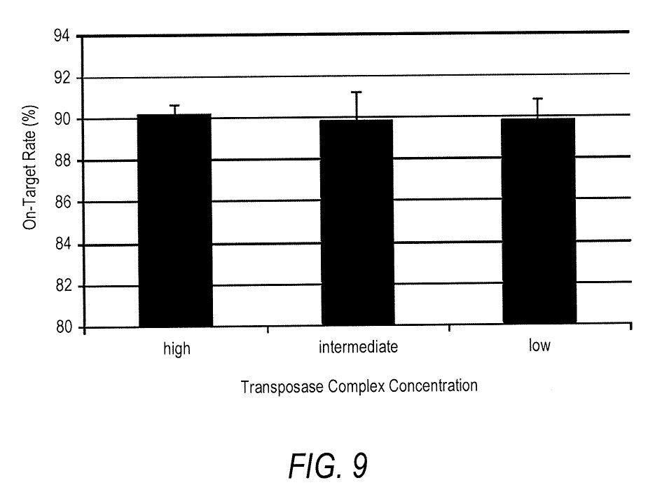

The effects of the concentration of transposase complex on the final insert size distribution, as well as metrics including on-target rate, were investigated by incubating 50 ng of human genomic DNA (Coriell, NA24385) with increasing quantities of transposase complex. Specifically, 50 ng of human genomic DNA (Coriell, NA24385) were incubated with one of a high concentration (36 μg/mL), an intermediate concentration (9 μg/mL), or a low concentration (1.125 μg/mL) of transposase complex. The highest concentration of transposase complex resulted in a greater abundance of shorter nucleic acid fragments (having an average fragment size of 550 nucleotides), whereas the lowest concentration of transposase complex resulted in larger nucleic acid fragments (having an average fragment size of 614 nucleotides). The intermediate concentration of transposase complex resulted in an average fragment size of 580 nucleotides. Moreover, nucleic acid libraries prepared with three different transposase complex concentrations exhibited similar on-target rates with no significant difference between the three concentrations (FIG. 9), which was indicative that the on-target rate is independent of insert size within the range tested.

### Example 3

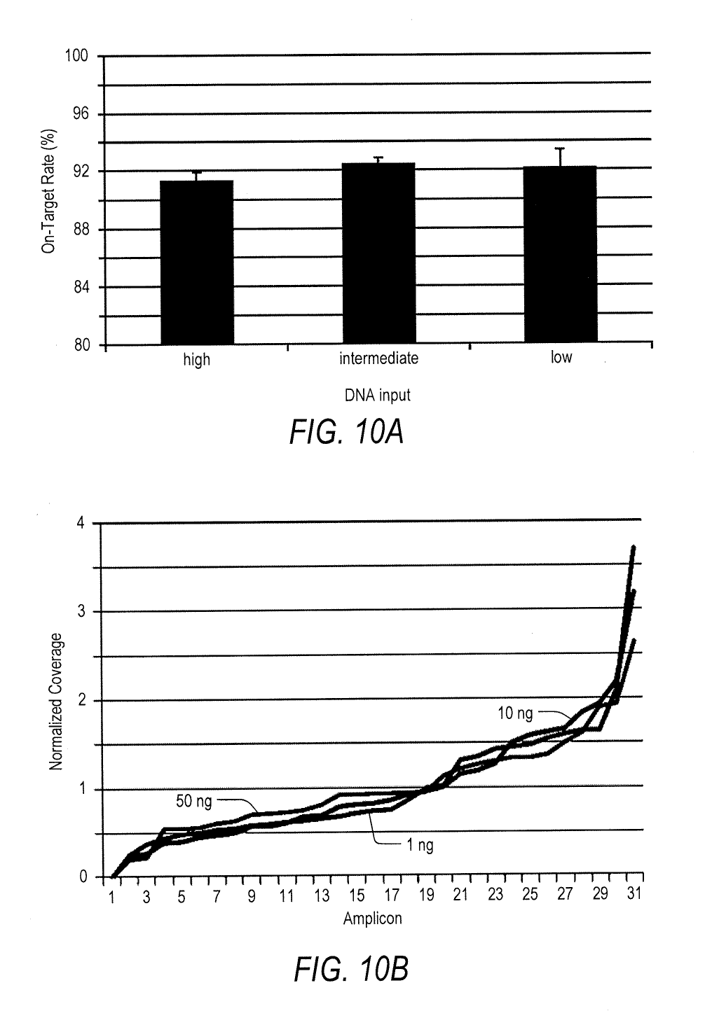

Turning to FIGS. 10A and 10B, the impact of lowering the DNA input into the TnPrep workflow was investigated using the Quantitative Multiplex Reference Standard (HORIZON DISCOVERY HD701). Similar on-target rates were observed with decreasing DNA input from 50 ng to 1 ng, with all on-target rates calculated to be greater than 90% (in accordance with FIG. 10A, the high level of DNA input corresponds to 50 ng, the intermediate level of DNA input corresponds to 10 ng and the low level of DNA input corresponds to 1 ng). Notably, coverage uniformity was unaffected by decreasing DNA input. As shown in Table 7, the percentage of bases covered at a depth of at least 0.2× of the mean was greater than 94% for each of the tested DNA input amounts.

TABLE 7

| DNA input (ng) | Bases covered at ≧0.2x of mean (%) |
|---|---|
| 50 | 94.2 |
| 10 | 96.1 |
| 1 | 94.9 |

### Example 4

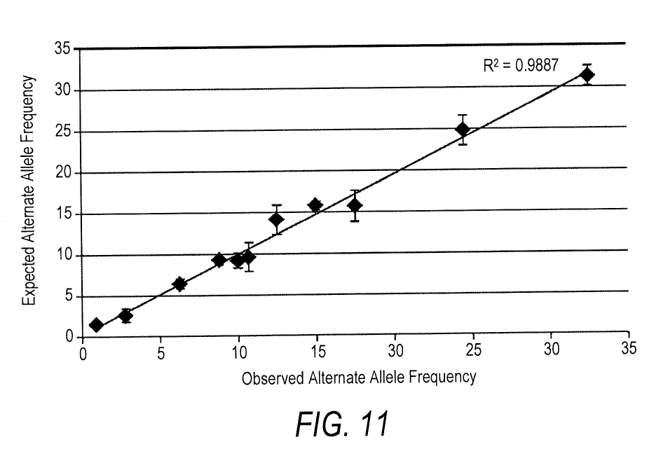

To investigate the ability of the disclosed methods to detect mutations, 11 single nucleotide variants (SNVs) and 2 deletions present in a Quantitative Multiplex Reference Standard (HORIZON DISCOVERY HD701) were targeted by a primer panel. The SNVs and deletions are known to be present at frequencies ranging from 0.9% up to 32.5%. Libraries were also constructed from a Structural Reference Standard (HORIZON DISCOVERY HD753), containing 2 gene fusions. With reference to FIG. 11, there was a strong correlation (R²=0.9887) between observed and expected allele frequencies in the 50 ng libraries prepared from the Quantitative Multiplex Reference Standard (HORIZON DISCOVERY HD701). The libraries prepared from 10 ng of the Quantitative Multiplex Reference Standard (HORIZON DISCOVERY HD701) exhibited a similarly strong correlation between observed and expected allele frequencies, while the 1 ng libraries performed slightly worse (data not shown). Variants were detected using a previously published method (Wilm A. et al., 2012. Nucleic Acids Res. December; 40(22):11189-201).

### Example 5

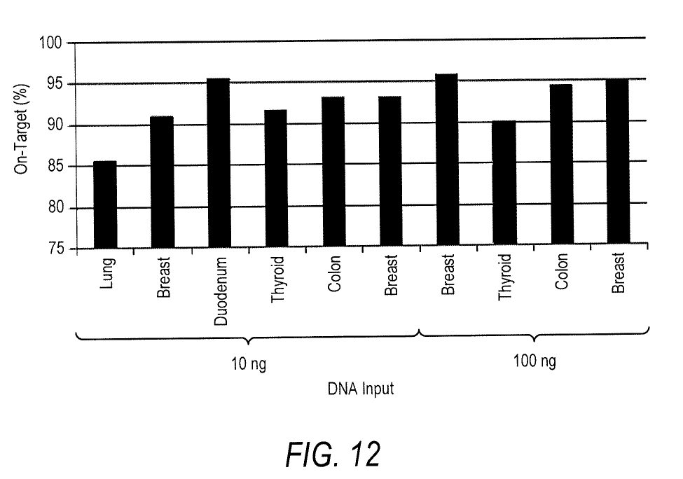

The performance of the disclosed method was further tested on a range of formalin-fixed paraffin embedded tissue (FFPET) samples from different tissue types and of varying quality. With reference to FIG. 12, on-target rates for nucleic acid libraries constructed from FFPE DNA were generally observed to be at least 90%, with the exception of a single library. No substantial difference in on-target rates was observed for libraries prepared from 10 ng of FFPE DNA as compared to libraries prepared from 100 ng of FFPE DNA. Notably, libraries derived from high quality DNA exhibited greater coverage uniformity than libraries constructed from FFPE (see Table 8).

TABLE 8

| Tissue Sample | Bases covered at ≧0.2x of mean (%) |
|---|---|
| Colon | 72.4 |
| Breast | 81.8 |
| Lung | 84.9 |
| Breast | 70.3 |
| Thyroid | 79.3 |

### Example 6

In this example, molecular barcodes (UIDs) were incorporated into the R1 arm in place of the i5 index as shown for the standard-length arm (standard R1 (SEQ ID NO:10) and complementary sequence (SEQ ID NO: 11); 15 Primer (SEQ ID NO:12, with index sequence underlined)) as well as for the long R1 arm (long R1 arm (SEQ ID NO: 13, with UID sequence underlined and bolded) and complementary sequence (SEQ ID NO: 14); 15 Primer (SEQ ID NO:15)):

Standard R1
(5′ to 3′) =
TCGTCGGCAGCGTCAGATGTGTATAAGAGACAG
(3′ to 5′) =
TCTACACATATTCTCTGTC
I5 Primer
(5′ to 3′) =
AATGATACGGCGACCACCGAGATCTACACTAGATCGCTCGTCG
GCAGCGTC

Long Arm R1 with UID in Place of i5 Index:

Long R1 Arm
(5′ to 3′) =
GACCACCGAGATCTACACNNNNNNNNTCGTCGGCAGCGTCAGA
TGTGTATAAGAGACAG
(3′ to 5′) =
TCTACACATATTCTCTGTC
I5 Primer
(5′ to 3′) =
AATGATACGGCGACCACCGAGATCTACAC

UID sequences of the disclosure may comprise or consist of 8 bases (e.g. 8 degenerate bases), which are preferably incorporated into one or both arms of the transposase. Preferably, the UID sequence replaces an index sequence (e.g. an i5 index sequence), as shown above. The incorporation of a UID sequence into an arm of the transposase facilitates the resolution of duplicates, enabling the generation of a consensus sequence for each original fragment of the resultant library. Generation of a consensus sequence for each original fragment of the resultant library using the UID sequences enables detection of true and/or rare variants in the library with an increased sensitivity compared to the use of a transposase lacking the UID sequences or a method lacking the consensus sequence for each original fragment of the resultant library.

For the above example and a final transposase concentration of 7.2 μg/mL, libraries were prepared with pre-loaded transposase containing only R1 arms from 10 ng Coriell DNA (NA12878) as shown in Table 9.

TABLE 9

| Component | Volume (μL) | Final Concentration | |
|---|---|---|---|
| DNA | 4 | 2.5 | ng/μL |
| Reaction buffer | 10 | 1x | |
| MnCl₂ | 2 | 2 | mM |
| Transposase (0.036 mg/ml) | 4 | 7.2 | μg/mL |

| | | |
|---|---|---|
| Total | 20 | n/a |

Samples were vortexed, spun down and incubated at 55° C. for 5 min.

After incubation, 20 μL GHCl stop solution was added, and the samples were again vortexed, centrifuged and incubated at room temperature for 5 min.

Clean up reactions were carried out with 40 μL SPRI® beads and samples were resuspended to a total volume of 11 μL.

Some samples were then heat treated at 95° C. for 5 min to denature strands and prevent nick translation in the subsequent amplification step. Alternatively, some samples were not heat-treated and a nick-translation step was added to the subsequent amplification step. In some embodiments, the nick-translation step comprises an incubation of the amplification reaction at 72° C. for 3 minutes. The inclusion of the nick-translation step produces an amplification product having an adapter on both the 5′ and 3′ ends of the fragments, as opposed to only the 5′ end of each fragment in the absence of the nick-translation step.

An amplification reaction was set up as shown in Table 10, and samples were cycled in a thermal cycler according to the protocol shown in Table 3.

| Component | Volume (μL) |
|---|---|
| DNA | 10 |
| 5x ReadyMix | 4 |
| i5 primer (2.5 μM) | 2 |
| Reverse primers (1 μM) | 2 |
| Total | 20 |

After amplification, samples were cleaned up with 40 μL SPRI beads and resuspended to a final volume of 11 μL. Samples were prepared for a second round of amplification as described in Table 11, and cycled for 16 cycles in a thermal cycler according to the protocol shown in Table 5.

| Component | Volume (μL) |
|---|---|
| PCR product | 10 |
| Hifi HotStart ReadyMix | 25 |
| P1 primer (2.5 μM) | 5 |
| Reverse primers (2.5 μM) | 2 |
| I7 primer (2.5 μM) | 5 |
| Total | 50 |

Amplification Primers (Wherein the (*) in a Primer Sequence Represents a Phosphorothioate Bond):

I5 primer:
(SEQ ID NO: 15)
AATGATACGGCGACCACCGAGATCTACAC
P1 primer:
(SEQ ID NO: 16)
AAT GAT ACG GCG ACC ACC G*A

The 31 outer target specific reverse primers were as follows:

| | |
|---|---|
| CTTCTCCACTTAATAAGCAGTTGA*T | SEQ ID NO: 3 |
| TAAGGTATCAATAAATACTCACCAATCTT*C | SEQ ID NO: 4 |
| CCAGGCTGCCTCACCA*T | SEQ ID NO: 5 |
| CAGAAATCCTAAATGGTGGAGT*C | SEQ ID NO: 17 |
| GGCTTGGGCAAAGGAAAT*A | SEQ ID NO: 18 |
| CAAGGAAGCAGGACACCAA*T | SEQ ID NO: 19 |
| TCAAACATCATCTTGTGAAAC*A | SEQ ID NO: 20 |
| CAGTTGGCTTACTGGAAGTTG*A | SEQ ID NO: 21 |
| AATCGGTTTAGGAATACAATTCT*G | SEQ ID NO: 22 |
| CCAGAATTATAGGAACTTGCTAACA*G | SEQ ID NO: 23 |
| GGTGGAGGTAATTTTGAAGC*A | SEQ ID NO: 24 |
| CAAGGGGAAAGTGTAAATCA*A | SEQ ID NO: 25 |
| CCCCATGGAACTTACCAAG*C | SEQ ID NO: 26 |
| AAAACCATCTTTCGTTTCCTT*C | SEQ ID NO: 27 |
| CATTTTGGCCAGGATGA*T | SEQ ID NO: 28 |
| TTCAAAGCCATTTTTCCAG*A | SEQ ID NO: 29 |
| GTGGGAAGGCGGTGTT*G | SEQ ID NO: 30 |
| CACAGCGTCTCCGAGTC*C | SEQ ID NO: 31 |
| GCACAGTTCAGAGGATATTTAAG*C | SEQ ID NO: 32 |
| GTGCAGCCCTCAGGGAG*T | SEQ ID NO: 33 |
| CACCCCCAGGATTCTTACAGAAAA*C | SEQ ID NO: 34 |
| CTGCCAGACATGAGAAAAGGTG*G | SEQ ID NO: 35 |
| AATATACAGCTTGCAAGGACTCTG*G | SEQ ID NO: 36 |
| CCAATATTGTCTTTGTGTTCCCGG*A | SEQ ID NO: 37 |
| TCTGCTTTATTTATTCCAATAGGTATGG*T | SEQ ID NO: 38 |
| AGTTGAAACTAAAAATCCTTTGCAG*G | SEQ ID NO: 39 |
| TAAACAATACAGCTAGTGGGAAGG*C | SEQ ID NO: 40 |
| AGTGTATTAACCTTATGTGTGACATG*T | SEQ ID NO: 41 |
| TGAGTGAAGGACTGAGAAAATCCC*T | SEQ ID NO: 42 |
| AGAAATTAGATCTCTTACCTAAACTCTTCA*T | SEQ ID NO: 43 |
| GTGGAATCCAGAGTGAGCTTTCAT*T | SEQ ID NO: 44 |

The 40 outer target specific reverse primers were as follows:

| | |
|---|---|
| GTTAATCAACTGATGCAAACTCTT*G | SEQ ID NO: 45 |
| AAATAATGCTCCTAGTACCTGTAG*A | SEQ ID NO: 46 |
| TCCTTTAATACAGAATATGGGTAAAGA*T | SEQ ID NO: 47 |
| TGTAAACCTTGCAGACAAACT*C | SEQ ID NO: 48 |
| CCATGAGGCAGAGCATAC*G | SEQ ID NO: 49 |
| ACATCCTGGTAGCTGAGG*G | SEQ ID NO: 50 |
| GTCTTTTGGTTTTTCTTGATAGTATTAAT*G | SEQ ID NO: 51 |
| TTCTTCCTAAGTGCAAAAGATAAC*T | SEQ ID NO: 52 |
| CAGTGTTTCTTTTAAATACCTGTTAAGTT*T | SEQ ID NO: 53 |
| CCACAGTTGCACAATATCCTT*T | SEQ ID NO: 54 |
| TGCAGCAATTCACTGTAAAG*C | SEQ ID NO: 55 |
| CTAATGTATATATGTTCTTAAATGGCTAC*G | SEQ ID NO: 56 |
| CCAGGACCAGAGGAAACC*T | SEQ ID NO: 57 |
| AAATAGTTTAAGATGAGTCATATTTGTG*G | SEQ ID NO: 58 |
| CTGTGGGGTGGAGAGCT*G | SEQ ID NO: 59 |
| GGTCACACTTGTTCCCCA*C | SEQ ID NO: 60 |
| CCGTTTGATCTGCTCCC*T | SEQ ID NO: 61 |
| CTGGATGGTCAGCGCAC*T | SEQ ID NO: 62 |
| GTTATGATTTTGCAGAAAACAGATC*T | SEQ ID NO: 63 |
| ACTATAATTACTCCTTAATGTCAGCTTA*T | SEQ ID NO: 64 |
| TGAAAATGGTCAGAGAAACCTTTAT*C | SEQ ID NO: 65 |
| TGCAGTTGTTTACCATGATAAC*G | SEQ ID NO: 66 |
| CGAAATAACACAAATTTTTAAGGTTACTG*A | SEQ ID NO: 67 |
| TGATTACACAGTATCCTCGACA*T | SEQ ID NO: 68 |
| CTGAAAAGAGTGAAGGATATAGGATA*C | SEQ ID NO: 69 |
| ACTGTTCTTCCTCAGACATTC*A | SEQ ID NO: 70 |
| CCACAGAGAAGTTGTTGAGG*G | SEQ ID NO: 71 |
| GCCTTGTACTGCAGAGACA*A | SEQ ID NO: 72 |
| TGGTGATGGGCTTGGTC*C | SEQ ID NO: 73 |
| TACAGCGGAGAAGGGAGC*G | SEQ ID NO: 74 |
| CAGGTAGGATCCAGCCC*A | SEQ ID NO: 75 |
| ATCCCCCAAAGCCAACA*A | SEQ ID NO: 76 |
| GTTTGGGGGTGTGTGGT*C | SEQ ID NO: 77 |
| GGTGCTTCCCATTCCAG*G | SEQ ID NO: 78 |
| ACAGTCAAGAAGAAAACGG*C | SEQ ID NO: 79 |
| AGAGGAGCTGGTGTTGTT*G | SEQ ID NO: 80 |
| ACCGCTTCTTGTCCTGC*T | SEQ ID NO: 81 |
| GAGCAGTAAGGAGATTCCC*C | SEQ ID NO: 82 |
| AAATCGGTAAGAGGTGGG*C | SEQ ID NO: 83 |
| CCCCCTACTGCTCACCT*G | SEQ ID NO: 84 |

The 31 target specific reverse primers with out-nested portion of adapter sequence were as follows:

| | |
|---|---|
| GTCTCGTGGGCTCGGAGATGTGTATAAGAGACA | SEQ ID NO: 7 |
| GAATTTTCAACTGCTTTACATAAGAAGCGT*T | |
| GTCTCGTGGGCTCGGAGATGTGTATAAGAGACA | SEQ ID NO: 6 |
| GTACCAGTGTGTTATTCAGGTAGGTC*A | |
| GTCTCGTGGGCTCGGAGATGTGTATAAGAGACA | SEQ ID NO: 8 |
| GGTCCTGACTGTGGCGTCA*T | |
| GTCTCGTGGGCTCGGAGATGTGTATAAGAGACA | SEQ ID NO: 85 |
| GAGAACAAATTCCTTTGTTATGCAGAC*A | |
| GTCTCGTGGGCTCGGAGATGTGTATAAGAGACA | SEQ ID NO: 86 |
| GTTGCCAATATTTAACCAATTTTGACCTAC*A | |
| GTCTCGTGGGCTCGGAGATGTGTATAAGAGACA | SEQ ID NO: 87 |
| GCAAGAAGATGCTCTGAGTCTAATGAAGT*T | |
| GTCTCGTGGGCTCGGAGATGTGTATAAGAGACA | SEQ ID NO: 88 |
| GGGTCTATAATCCAGATGATTCTTTAACAG*G | |
| GTCTCGTGGGCTCGGAGATGTGTATAAGAGACA | SEQ ID NO: 89 |
| GCTTTGTCCACCTGGAACTTGG*T | |
| GTCTCGTGGGCTCGGAGATGTGTATAAGAGACA | SEQ ID NO: 90 |
| GCTCACTCTAACAAGCAGATAACTTTCACT*T | |
| GTCTCGTGGGCTCGGAGATGTGTATAAGAGACA | SEQ ID NO: 91 |
| GCAGAGAATGAGGGAGGAGTACATTAC*T | |
| GTCTCGTGGGCTCGGAGATGTGTATAAGAGACA | SEQ ID NO: 92 |
| GTTTTTGCTTTACGTGATGACTTTGTT*G | |
| GTCTCGTGGGCTCGGAGATGTGTATAAGAGACA | SEQ ID NO: 93 |
| GGCAAACCACAAAAGTATACTCCATGGTT*A | |
| GTCTCGTGGGCTCGGAGATGTGTATAAGAGACA | SEQ ID NO: 94 |
| GTCCATCTCTTGGAAACTCCCATC*T | |
| GTCTCGTGGGCTCGGAGATGTGTATAAGAGACA | SEQ ID NO: 95 |
| GAATGCAATGGATGATCTGGGAAATAAG*A | |
| GTCTCGTGGGCTCGGAGATGTGTATAAGAGACA | SEQ ID NO: 96 |
| GTGATGCCTTGACCTCCTGATC*T | |
| GTCTCGTGGGCTCGGAGATGTGTATAAGAGACA | SEQ ID NO: 97 |
| GACTAGAGTGTCTGTGTAATCAAACAAGTT*T | |
| GTCTCGTGGGCTCGGAGATGTGTATAAGAGACA | SEQ ID NO: 98 |
| GCAGGCGTCCTACTGGCA*T | |
| GTCTCGTGGGCTCGGAGATGTGTATAAGAGACA | SEQ ID NO: 99 |
| GCTCCTTCACCTTGCCGTAAGA*G | |
| GTCTCGTGGGCTCGGAGATGTGTATAAGAGACA | SEQ ID NO: 100 |
| GGACATCTCCATTCTTCTCTTTTAATTGC*C | |
| GTCTCGTGGGCTCGGAGATGTGTATAAGAGACA | SEQ ID NO: 101 |
| GCCTAGCACGTGCCTACC*T | |
| GTCTCGTGGGCTCGGAGATGTGTATAAGAGACA | SEQ ID NO: 102 |
| GTGGTGAAACCTGTTTGTTGGACAT*A | |
| GTCTCGTGGGCTCGGAGATGTGTATAAGAGACA | SEQ ID NO: 103 |
| GCACAGCAAAGCAGAAACTCACAT*C | |
| GTCTCGTGGGCTCGGAGATGTGTATAAGAGACA | SEQ ID NO: 104 |
| GTGTGCCAGGGACCTTACCTTA*T | |
| GTCTCGTGGGCTCGGAGATGTGTATAAGAGACA | SEQ ID NO: 105 |
| GATAGTCCAGGAGGCAGCCGA*A | |
| GTCTCGTGGGCTCGGAGATGTGTATAAGAGACA | SEQ ID NO: 106 |
| GATGCTGAGATCAGCCAAATTCAGTT*A | |
| GTCTCGTGGGCTCGGAGATGTGTATAAGAGACA | SEQ ID NO: 107 |
| GGTCAAGCAGAGAATGGGTACTC*A | |
| GTCTCGTGGGCTCGGAGATGTGTATAAGAGACA | SEQ ID NO: 108 |
| GCAGGAAAATGCTGGCTGACCT*A | |
| GTCTCGTGGGCTCGGAGATGTGTATAAGAGACA | SEQ ID NO: 109 |
| GTAAGGCCTGCTGAAAATGACTGA*A | |
| GTCTCGTGGGCTCGGAGATGTGTATAAGAGACA | SEQ ID NO: 110 |
| GAGGACTTGGGAGGTATCCACA*T | |
| GTCTCGTGGGCTCGGAGATGTGTATAAGAGACA | SEQ ID NO: 111 |
| GCTTGCTCTGATAGGAAAATGAGATCTACT*G | |
| GTCTCGTGGGCTCGGAGATGTGTATAAGAGACA | SEQ ID NO: 112 |
| GTGTGGAAGATCCAATCCATTTTTGTT*G | |

The 40 target specific reverse primers with out-nested portion of the adapter sequence were as follows:

| | |
|---|---|
| GTCTCGTGGGCTCGGAGATGTGTATAAGAGACA | SEQ ID NO: 113 |
| GTGAAAGCTGTACCATACCTG*T | |
| GTCTCGTGGGCTCGGAGATGTGTATAAGAGACA | SEQ ID NO: 114 |
| GAGGTTAATATCCGCAAATGACTT*G | |
| GTCTCGTGGGCTCGGAGATGTGTATAAGAGACA | SEQ ID NO: 115 |
| GAGATGATCCGACAAGTGAGA*G | |
| GTCTCGTGGGCTCGGAGATGTGTATAAGAGACA | SEQ ID NO: 116 |
| GTACGTCTCCTCCGACCA*C | |
| GTCTCGTGGGCTCGGAGATGTGTATAAGAGACA | SEQ ID NO: 117 |
| GCGCAGCCTGTACCCAGT*G | |
| GTCTCGTGGGCTCGGAGATGTGTATAAGAGACA | SEQ ID NO: 118 |
| GGCGGAAGATGAAGATTTCGGA*T | |
| GTCTCGTGGGCTCGGAGATGTGTATAAGAGACA | SEQ ID NO: 119 |
| GGTTAGCTCATTTTTGTTAATGGTG*G | |
| GTCTCGTGGGCTCGGAGATGTGTATAAGAGACA | SEQ ID NO: 120 |
| GTTAAACTTTTCTTTTAGTTGTGCTG*A | |
| GTCTCGTGGGCTCGGAGATGTGTATAAGAGACA | SEQ ID NO: 121 |
| GGTATGCAACATTTCTAAAGTTACCTA*C | |
| GTCTCGTGGGCTCGGAGATGTGTATAAGAGACA | SEQ ID NO: 122 |
| GAAGACCATAACCCACCACA*G | |
| GTCTCGTGGGCTCGGAGATGTGTATAAGAGACA | SEQ ID NO: 123 |
| GCTGGAAAGGGACGAACTGG*T | |
| GTCTCGTGGGCTCGGAGATGTGTATAAGAGACA | SEQ ID NO: 124 |
| GCGACCCAGTTACCATAGCA*A | |
| GTCTCGTGGGCTCGGAGATGTGTATAAGAGACA | SEQ ID NO: 125 |
| GAGAAAATGGAAGTCTATGTGATCAA*G | |
| GTCTCGTGGGCTCGGAGATGTGTATAAGAGACA | SEQ ID NO: 126 |
| GCATTTTAAATTTTCTTTCTCTAGGTGAA*G | |
| GTCTCGTGGGCTCGGAGATGTGTATAAGAGACA | SEQ ID NO: 127 |
| GGGTCTTGGCCGAGGTCT*C | |
| GTCTCGTGGGCTCGGAGATGTGTATAAGAGACA | SEQ ID NO: 128 |
| GCAGCACCATGGGCACGT*C | |
| GTCTCGTGGGCTCGGAGATGTGTATAAGAGACA | SEQ ID NO: 129 |
| GGAGAGGTGGAAAGCGAGA*G | |
| GTCTCGTGGGCTCGGAGATGTGTATAAGAGACA | SEQ ID NO: 130 |
| GCACTCTTGCCCACACCG*C | |
| GTCTCGTGGGCTCGGAGATGTGTATAAGAGACA | SEQ ID NO: 131 |
| GCTGTATTTATTTCAGTGTTACTTACCT*G | |
| GTCTCGTGGGCTCGGAGATGTGTATAAGAGACA | SEQ ID NO: 132 |
| GTTATATTCAATTTAAACCCACCTATAATG*G | |
| GTCTCGTGGGCTCGGAGATGTGTATAAGAGACA | SEQ ID NO: 133 |
| GGTATCAAAGAATGGTCCTGCA*C | |
| GTCTCGTGGGCTCGGAGATGTGTATAAGAGACA | SEQ ID NO: 134 |
| GACACAACACAAAATAGCCGTAT*A | |
| GTCTCGTGGGCTCGGAGATGTGTATAAGAGACA | SEQ ID NO: 135 |
| GTCTATTCTTTCCTTTGTAGTGTCC*A | |
| GTCTCGTGGGCTCGGAGATGTGTATAAGAGACA | SEQ ID NO: 136 |
| GTGATTTCTGTTTTTACCTCCTAAAGA*A | |
| GTCTCGTGGGCTCGGAGATGTGTATAAGAGACA | SEQ ID NO: 137 |
| GATCTTTAAAGAGAAATTTGCTAAAGCTGT*G | |
| GTCTCGTGGGCTCGGAGATGTGTATAAGAGACA | SEQ ID NO: 138 |
| GACGTGTTTTGATCAAAGAAGAG*G | |
| GTCTCGTGGGCTCGGAGATGTGTATAAGAGACA | SEQ ID NO: 139 |
| GAGCCTCACGTTGGTCCA*C | |
| GTCTCGTGGGCTCGGAGATGTGTATAAGAGACA | SEQ ID NO: 140 |
| GGGATGGCTAGGCGAGGA*G | |
| GTCTCGTGGGCTCGGAGATGTGTATAAGAGACA | SEQ ID NO: 141 |
| GAGGGACTAGGCGTGGGA*T | |
| GTCTCGTGGGCTCGGAGATGTGTATAAGAGACA | SEQ ID NO: 142 |
| GCCAAGCCCTAGGGTGGT*G | |
| GTCTCGTGGGCTCGGAGATGTGTATAAGAGACA | SEQ ID NO: 143 |
| GACGCTCTTCTCACTCATATC*C | |
| GTCTCGTGGGCTCGGAGATGTGTATAAGAGACA | SEQ ID NO: 144 |
| GAAGAAATCTTAGACGTAAGCCCCT*C | |
| GTCTCGTGGGCTCGGAGATGTGTATAAGAGACA | SEQ ID NO: 145 |
| GTCCCATACCCTCTCAGC*G | |
| GTCTCGTGGGCTCGGAGATGTGTATAAGAGACA | SEQ ID NO: 146 |
| GGGATGAGCTACCTGGAGGA*T | |
| GTCTCGTGGGCTCGGAGATGTGTATAAGAGACA | SEQ ID NO: 147 |
| GATTTTGAGTGTTAGACTGGAAAC*T | |
| GTCTCGTGGGCTCGGAGATGTGTATAAGAGACA | SEQ ID NO: 148 |
| GGGGCAGTGCTAGGAAAGA*G | |
| GTCTCGTGGGCTCGGAGATGTGTATAAGAGACA | SEQ ID NO: 149 |
| GTGCTTACCTCGCTTAGTG*C | |
| GTCTCGTGGGCTCGGAGATGTGTATAAGAGACA | SEQ ID NO: 150 |
| GGCCGGGGATGTGATGAG*A | |
| GTCTCGTGGGCTCGGAGATGTGTATAAGAGACA | SEQ ID NO: 151 |
| GCAGGGGTCAGAGGCAAG*C | |
| GTCTCGTGGGCTCGGAGATGTGTATAAGAGACA | SEQ ID NO: 152 |
| GAGGGCCACTGACAACCA*C | |

The sequence of the NEXTERA amplification primer (N70×) was as follows (the I7 representing an index tag):

(SEQ ID NO: 9)
CAAGCAGAAGACGGCATACGAGAT(I7)GTCTCGTGGGCTCGG.

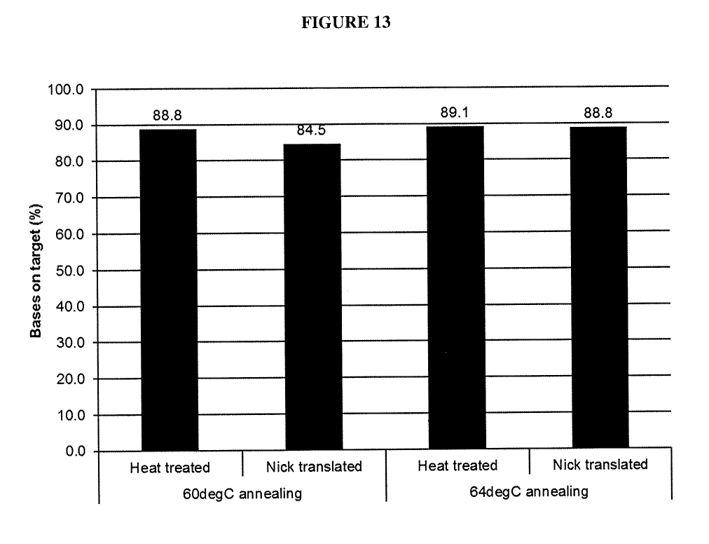

As shown in FIG. 13, the elimination of the heat denaturation step and inclusion of the nick-translation step maintained a comparable level of on-target amplification compared to the heat-denaturing step.

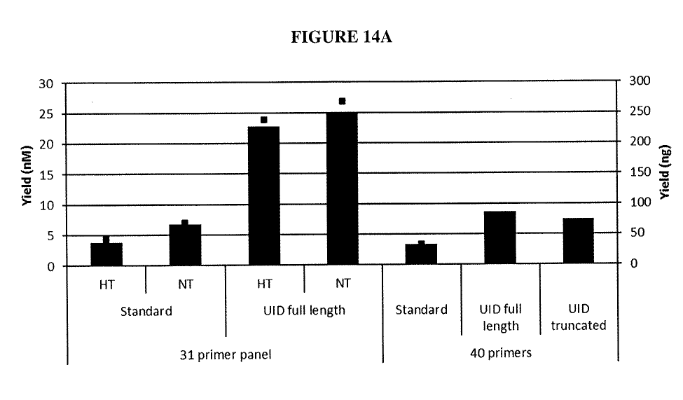

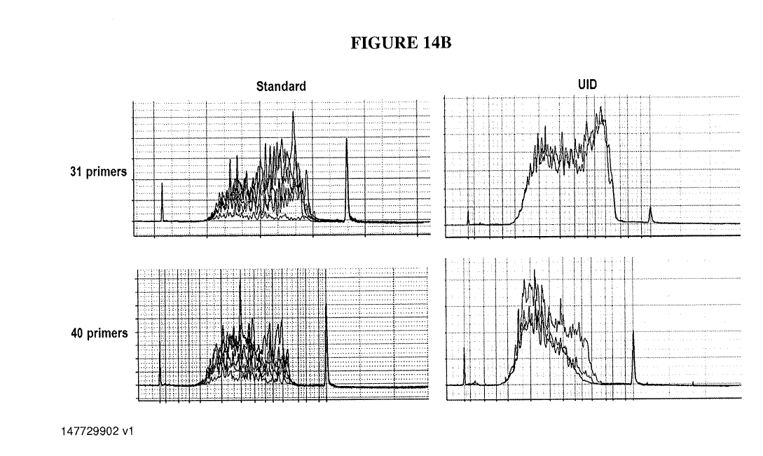

FIGS. 14A and 14B demonstrate that the including of the long R1 arm with the UID sequence may significantly improve yield compared to the use of a standard R1 arm.

### Other Embodiments

The schematic flow charts shown in the Figures are generally set forth as logical flow chart diagrams. As such, the depicted order and labeled steps are indicative of one embodiment of the presented method. Other steps and methods may be conceived that are equivalent in function, logic, or effect to one or more steps, or portions thereof, of the illustrated method. Additionally, the format and symbols employed in the Figures are provided to explain the logical steps of the method and are understood not to limit the scope of the method. Although various arrow types and line types may be employed, they are understood not to limit the scope of the corresponding method. Indeed, some arrows or other connectors may be used to indicate only the logical flow of the method. For instance, an arrow may indicate a waiting or monitoring period of unspecified duration between enumerated steps of the depicted method. Additionally, the order in which a particular method occurs may or may not strictly adhere to the order of the corresponding steps shown.

The present invention is presented in several varying embodiments in the following description with reference to the Figures, in which like numbers represent the same or similar elements. Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

The described features, structures, or characteristics of the invention may be combined in any suitable manner in one or more embodiments. In the following description, numerous specific details are recited to provide a thorough understanding of embodiments of the system. One skilled in the relevant art will recognize, however, that the system and method may both be practiced without one or more of the specific details, or with other methods, components, materials, and so forth. In other instances, well-known structures, materials, or operations are not shown or described in detail to avoid obscuring aspects of the invention. Accordingly, the foregoing description is meant to be exemplary, and does not limit the scope of present inventive concepts.

Each reference identified in the present application is herein incorporated by reference in its entirety.

What is claimed is:

1. A method for targeted enrichment of nucleic acids, the method comprising:
contacting a nucleic acid including at least one region of interest with a plurality of transposase complexes, each of the transposase complexes including at least a transposase and a first polynucleotide, the first polynucleotide having a transposon end sequence and a first label sequence, the transposon end sequence corresponding to the first transposase;
incubating the nucleic acid and the transposase complexes under conditions whereby multiple transposon end sequences and associated label sequences are inserted into the nucleic acid and the nucleic acid is fragmented into a plurality of nucleic acid fragments including first polynucleotide attached to each 5′ end of the nucleic acid fragments;
selectively amplifying the nucleic acid fragments, thereby enriching for a portion of the nucleic acid fragments including the at least one region of interest relative to a remaining portion of the nucleic acid fragments; and
sequencing the enriched nucleic acid fragments.

2. The method of claim 1, wherein selectively amplifying the nucleic acid fragments further includes a first round of PCR with at least one region specific primer complementary to the nucleic acid proximal to the at least one region of interest, and a label specific primer complementary to at least a portion of the first label sequence attached to each 5′ end of the nucleic acid fragments.

3. The method of claim 2, wherein selectively amplifying the nucleic acid fragments further includes at least ten region specific primers, wherein each of the region specific primers are complementary to the nucleic acid proximal to different regions of interest.

4. The method of claim 2, wherein selectively amplifying the nucleic acid fragments further includes at least 100 region specific primers, wherein each of the region specific primers are complementary to the nucleic acid proximal to different regions of interest.

5. The method of claim 2, wherein selectively amplifying the nucleic acid fragments further includes at least 1,000 region specific primers, wherein each of the region specific primers are complementary to the nucleic acid proximal to different regions of interest.

6. The method of claim 2, wherein selectively amplifying the nucleic acid fragments further includes at least 10,000 region specific primers, wherein each of the region specific primers are complementary to the nucleic acid proximal to different regions of interest.

7. The method of claim 2, wherein selectively amplifying the nucleic acid fragments further includes at least 100,000 region specific primers, wherein each of the region specific primers are complementary to the nucleic acid proximal to different regions of interest.

8. The method of claim 1, wherein the nucleic acid is a double-stranded DNA.

9. The method of claim 1, wherein the transposase complex includes two of the first polynucleotides.

10. The method of claim 2, wherein the at least one region specific primer includes a non-complementary 5′ tail sequence.

11. The method of claim 10, wherein the at least one label specific primer includes a non-complementary 5′ tail sequence.

12. The method of claim 11, wherein selectively amplifying the nucleic acid fragments further includes a second round of PCR with a first primer complementary to at least a portion of the region specific primer, and second primer complementary to at least a portion of the label specific primer.

13. The method of claim 12, wherein the at least one of the first primer and the second primer includes an adapter sequence.

14. The method of claim 1, wherein the first label sequence includes at least one of i) an adapter sequence, ii) a unique identifier sequence, and iii) and sample identifier sequence.

15. The method of claim 1, wherein the first label sequence includes an adapter sequence.

16. A kit for targeted enrichment of at least one region of interest within a nucleic acid, the kit comprising:
a transposase;
a first polynucleotide having a transposon end sequence and a first label sequence, the transposon end sequence corresponding to the first transposase;
at least one region specific primer complementary to a nucleic acid proximal to an at least one region of interest, and a label specific primer complementary to at least a portion of the first label sequence.

17. The kit of claim 16, further comprising a plurality of region specific primers.

18. The kit of claim 16, wherein the plurality of region specific primers includes at least 10 primers.

19. The kit of claim 16, wherein the plurality of region specific primers includes at least 100 primers.

20. The kit of claim 16, wherein the plurality of region specific primers includes at least 1,000 primers.

21. The kit of claim 16, wherein the plurality of region specific primers includes at least 10,000 primers.

22. The kit of claim 16, wherein the plurality of region specific primers includes at least 100,000 primers.

23. A kit for targeted enrichment of at least one region of interest within a nucleic acid, the kit comprising:
a transposase complex comprising a transposase and a first polynucleotide having a transposon end sequence and a first label sequence, the transposon end sequence corresponding to the first transposase;
at least one region specific primer complementary to a nucleic acid proximal to an at least one region of interest, and a label specific primer complementary to at least a portion of the first label sequence.

24. The kit of claim 23, wherein the transposase complex includes two of the first polynucleotides.

25. The kit of claim 24, further comprising a plurality of region specific primers

26. A method for targeted enrichment of nucleic acids, the method comprising:
contacting a nucleic acid including at least one region of interest with a plurality of transposase complexes, each of the transposase complexes including at least a transposase and a first polynucleotide, the first polynucleotide having a transposon end sequence and a first label sequence, the transposon end sequence corresponding to the first transposase;
incubating the nucleic acid and the transposase complexes under conditions whereby multiple transposon end sequences and associated label sequences are inserted into the nucleic acid and the nucleic acid is fragmented into a plurality of nucleic acid fragments including first polynucleotide attached to each 5′ end of the nucleic acid fragments;
performing a first round of amplification including the nucleic acid fragments and at least a first sequence specific primer that is out-nested relative to the region of interest, thereby yielding a first amplification product;
performing a second round of amplification including the first amplification product and at least a second sequence specific primer that is in-nested relative to first sequence specific primer, thereby yielding a second amplification product, thereby enriching for a portion of the nucleic acid fragments including the at least one region of interest relative to a remaining portion of the nucleic acid fragments; and
sequencing the enriched nucleic acid fragments including the second amplification product.

27. The method of claim 1, wherein the amplifying step comprises a denaturation step.

28. The method of claim 1, wherein the amplifying step does not comprise a denaturation step and wherein the amplifying step comprises a nick-translation step.

29. The method of claim 28, wherein the amplifying step produces a plurality of nucleic acid fragments including the first polynucleotide attached to each 5′ end of the nucleic acid fragments and the complement of the first polynucleotide attached to each 3′ end of the nucleic acid fragments.

30. The method of claim 26, wherein the first round of amplification comprises a denaturation step.

31. The method of claim 26, wherein the first round of amplification does not comprise a denaturation step and wherein the first round of amplification comprises a nick-translation step.

32. The method of claim 31, wherein the first round of amplification produces a plurality of nucleic acid fragments including the first polynucleotide attached to each 5′ end of the nucleic acid fragments and the complement of the first polynucleotide attached to each 3′ end of the nucleic acid fragments.


SEQUENCE LISTING (152 sequences)

SEQ ID NO: 1 - DNA, 33 bp, Artificial Sequence
  Description of Artificial Sequence Synthetic oligonucleotide
tcgtcggcagcgtcagatgtgtataagagacag

SEQ ID NO: 2 - DNA, 51 bp, Artificial Sequence
  Description of Artificial Sequence Synthetic primer
aatgatacggcgaccaccgagatctacacgcgtaagatcgtcggcagcgtc

SEQ ID NO: 3 - DNA, 25 bp, Artificial Sequence
  Description of Artificial Sequence Synthetic primer
cttctccacttaataagcagttgat

SEQ ID NO: 4 - DNA, 30 bp, Artificial Sequence
  Description of Artificial Sequence Synthetic primer
taaggtatcaataaatactcaccaatcttc

SEQ ID NO: 5 - DNA, 17 bp, Artificial Sequence
  Description of Artificial Sequence Synthetic primer
ccaggctgcctcaccat

SEQ ID NO: 6 - DNA, 60 bp, Artificial Sequence
  Description of Artificial Sequence Synthetic primer
gtctcgtgggctcggagatgtgtataagagacagtaccagtgtgttattcaggtaggtca

SEQ ID NO: 7 - DNA, 64 bp, Artificial Sequence
  Description of Artificial Sequence Synthetic primer
gtctcgtgggctcggagatgtgtataagagacagaattttcaactgctttacataagaagcgtt

SEQ ID NO: 8 - DNA, 53 bp, Artificial Sequence
  Description of Artificial Sequence Synthetic primer
gtctcgtgggctcggagatgtgtataagagacaggtcctgactgtggcgtcat

SEQ ID NO: 9 - DNA, 39 bp, Artificial Sequence
  Description of Artificial Sequence Synthetic primer
caagcagaagacggcatacgagatgtctcgtgggctcgg

SEQ ID NO: 10 - DNA, 33 bp, Artificial Sequence; identical to SEQ ID NO: 1
  Description of Artificial Sequence Synthetic oligonucleotide

SEQ ID NO: 11 - DNA, 19 bp, Artificial Sequence
  Description of Artificial Sequence Synthetic oligonucleotide
ctgtctcttatacacatct

SEQ ID NO: 12 - DNA, 51 bp, Artificial Sequence
  Description of Artificial Sequence Synthetic primer
aatgatacggcgaccaccgagatctacactagatcgctcgtcggcagcgtc

SEQ ID NO: 13 - DNA, 59 bp, Artificial Sequence
  Description of Artificial Sequence Synthetic oligonucleotide
  modified_base (19)..(26): a, c, t, g, unknown or other
gaccaccgagatctacacnnnnnnnntcgtcggcagcgtcagatgtgtataagagacag

SEQ ID NO: 14 - DNA, 19 bp, Artificial Sequence
  Description of Artificial Sequence Synthetic oligonucleotide
ctgtctcttatacacatct

SEQ ID NO: 15 - DNA, 29 bp, Artificial Sequence
  Description of Artificial Sequence Synthetic primer
aatgatacggcgaccaccgagatctacac

SEQ ID NO: 16 - DNA, 20 bp, Artificial Sequence
  Description of Artificial Sequence Synthetic primer
aatgatacggcgaccaccga

SEQ ID NO: 17 - DNA, 23 bp, Artificial Sequence
  Description of Artificial Sequence Synthetic primer
cagaaatcctaaatggtggagtc

SEQ ID NO: 18 - DNA, 19 bp, Artificial Sequence
  Description of Artificial Sequence Synthetic primer
ggcttgggcaaaggaaata

SEQ ID NO: 19 - DNA, 20 bp, Artificial Sequence
  Description of Artificial Sequence Synthetic primer
caaggaagcaggacaccaat

SEQ ID NO: 20 - DNA, 22 bp, Artificial Sequence
  Description of Artificial Sequence Synthetic primer
tcaaacatcatcttgtgaaaca

SEQ ID NO: 21 - DNA, 22 bp, Artificial Sequence
  Description of Artificial Sequence Synthetic primer
cagttggcttactggaagttga

SEQ ID NO: 22 - DNA, 24 bp, Artificial Sequence
  Description of Artificial Sequence Synthetic primer
aatcggtttaggaatacaattctg

SEQ ID NO: 23 - DNA, 26 bp, Artificial Sequence
  Description of Artificial Sequence Synthetic primer
ccagaattataggaacttgctaacag

SEQ ID NO: 24 - DNA, 21 bp, Artificial Sequence
  Description of Artificial Sequence Synthetic primer
ggtggaggtaattttgaagca

SEQ ID NO: 25 - DNA, 21 bp, Artificial Sequence
  Description of Artificial Sequence Synthetic primer
caaggggaaagtgtaaatcaa

SEQ ID NO: 26 - DNA, 20 bp, Artificial Sequence
  Description of Artificial Sequence Synthetic primer
ccccatggaacttaccaagc

SEQ ID NO: 27 - DNA, 22 bp, Artificial Sequence
  Description of Artificial Sequence Synthetic primer
aaaaccatctttcgtttccttc

SEQ ID NO: 28 - DNA, 18 bp, Artificial Sequence
  Description of Artificial Sequence Synthetic primer
cattttggccaggatgat

SEQ ID NO: 29 - DNA, 20 bp, Artificial Sequence
  Description of Artificial Sequence Synthetic primer
ttcaaagccatttttccaga

SEQ ID NO: 30 - DNA, 17 bp, Artificial Sequence
  Description of Artificial Sequence Synthetic primer
gtgggaaggcggtgttg

SEQ ID NO: 31 - DNA, 18 bp, Artificial Sequence
  Description of Artificial Sequence Synthetic primer
cacagcgtctccgagtcc

SEQ ID NO: 32 - DNA, 24 bp, Artificial Sequence
  Description of Artificial Sequence Synthetic primer
gcacagttcagaggatatttaagc

SEQ ID NO: 33 - DNA, 18 bp, Artificial Sequence
  Description of Artificial Sequence Synthetic primer
gtgcagccctcagggagt

SEQ ID NO: 34 - DNA, 25 bp, Artificial Sequence
  Description of Artificial Sequence Synthetic primer
cacccccaggattcttacagaaaac

SEQ ID NO: 35 - DNA, 23 bp, Artificial Sequence
  Description of Artificial Sequence Synthetic primer
ctgccagacatgagaaaaggtgg

SEQ ID NO: 36 - DNA, 25 bp, Artificial Sequence
  Description of Artificial Sequence Synthetic primer
aatatacagcttgcaaggactctgg

SEQ ID NO: 37 - DNA, 25 bp, Artificial Sequence
  Description of Artificial Sequence Synthetic primer
ccaatattgtctttgtgttcccgga

SEQ ID NO: 38 - DNA, 29 bp, Artificial Sequence
  Description of Artificial Sequence Synthetic primer
tctgctttatttattccaataggtatggt

SEQ ID NO: 39 - DNA, 26 bp, Artificial Sequence
  Description of Artificial Sequence Synthetic primer
agttgaaactaaaaatcctttgcagg

SEQ ID NO: 40 - DNA, 25 bp, Artificial Sequence
  Description of Artificial Sequence Synthetic primer
taaacaatacagctagtgggaaggc

SEQ ID NO: 41 - DNA, 27 bp, Artificial Sequence
  Description of Artificial Sequence Synthetic primer
agtgtattaaccttatgtgtgacatgt

SEQ ID NO: 42 - DNA, 25 bp, Artificial Sequence
  Description of Artificial Sequence Synthetic primer
tgagtgaaggactgagaaaatccct

SEQ ID NO: 43 - DNA, 31 bp, Artificial Sequence
  Description of Artificial Sequence Synthetic primer
agaaattagatctcttacctaaactcttcat

SEQ ID NO: 44 - DNA, 25 bp, Artificial Sequence
  Description of Artificial Sequence Synthetic primer
gtggaatccagagtgagctttcatt

SEQ ID NO: 45 - DNA, 25 bp, Artificial Sequence
  Description of Artificial Sequence Synthetic primer
gttaatcaactgatgcaaactcttg

SEQ ID NO: 46 - DNA, 25 bp, Artificial Sequence
  Description of Artificial Sequence Synthetic primer
aaataatgctcctagtacctgtaga

SEQ ID NO: 47 - DNA, 28 bp, Artificial Sequence
  Description of Artificial Sequence Synthetic primer
tcctttaatacagaatatgggtaaagat

SEQ ID NO: 48 - DNA, 22 bp, Artificial Sequence
  Description of Artificial Sequence Synthetic primer
tgtaaaccttgcagacaaactc

SEQ ID NO: 49 - DNA, 19 bp, Artificial Sequence
  Description of Artificial Sequence Synthetic primer
ccatgaggcagagcatacg

SEQ ID NO: 50 - DNA, 19 bp, Artificial Sequence
  Description of Artificial Sequence Synthetic primer
acatcctggtagctgaggg

SEQ ID NO: 51 - DNA, 30 bp, Artificial Sequence
  Description of Artificial Sequence Synthetic primer
gtcttttggtttttcttgatagtattaatg

SEQ ID NO: 52 - DNA, 25 bp, Artificial Sequence
  Description of Artificial Sequence Synthetic primer
ttcttcctaagtgcaaaagataact

SEQ ID NO: 53 - DNA, 30 bp, Artificial Sequence
  Description of Artificial Sequence Synthetic primer
cagtgtttcttttaaatacctgttaagttt

SEQ ID NO: 54 - DNA, 22 bp, Artificial Sequence
  Description of Artificial Sequence Synthetic primer
ccacagttgcacaatatccttt

SEQ ID NO: 55 - DNA, 21 bp, Artificial Sequence
  Description of Artificial Sequence Synthetic primer
tgcagcaattcactgtaaagc

SEQ ID NO: 56 - DNA, 30 bp, Artificial Sequence
  Description of Artificial Sequence Synthetic primer
ctaatgtatatatgttcttaaatggctacg

SEQ ID NO: 57 - DNA, 19 bp, Artificial Sequence
  Description of Artificial Sequence Synthetic primer
ccaggaccagaggaaacct

SEQ ID NO: 58 - DNA, 29 bp, Artificial Sequence
  Description of Artificial Sequence Synthetic primer
aaatagtttaagatgagtcatatttgtgg

SEQ ID NO: 59 - DNA, 18 bp, Artificial Sequence
  Description of Artificial Sequence Synthetic primer
ctgtggggtggagagctg

SEQ ID NO: 60 - DNA, 19 bp, Artificial Sequence
  Description of Artificial Sequence Synthetic primer
ggtcacacttgttccccac

SEQ ID NO: 61 - DNA, 18 bp, Artificial Sequence
  Description of Artificial Sequence Synthetic primer
ccgtttgatctgctccct

SEQ ID NO: 62 - DNA, 18 bp, Artificial Sequence
  Description of Artificial Sequence Synthetic primer
ctggatggtcagcgcact

SEQ ID NO: 63 - DNA, 26 bp, Artificial Sequence
  Description of Artificial Sequence Synthetic primer
gttatgattttgcagaaaacagatct

SEQ ID NO: 64 - DNA, 29 bp, Artificial Sequence
  Description of Artificial Sequence Synthetic primer
actataattactccttaatgtcagcttat

SEQ ID NO: 65 - DNA, 26 bp, Artificial Sequence
  Description of Artificial Sequence Synthetic primer
tgaaaatggtcagagaaacctttatc

SEQ ID NO: 66 - DNA, 23 bp, Artificial Sequence
  Description of Artificial Sequence Synthetic primer
tgcagttgtttaccatgataacg

SEQ ID NO: 67 - DNA, 30 bp, Artificial Sequence
  Description of Artificial Sequence Synthetic primer
cgaaataacacaaatttttaaggttactga

SEQ ID NO: 68 - DNA, 23 bp, Artificial Sequence
  Description of Artificial Sequence Synthetic primer
tgattacacagtatcctcgacat

SEQ ID NO: 69 - DNA, 27 bp, Artificial Sequence
  Description of Artificial Sequence Synthetic primer
ctgaaaagagtgaaggatataggatac

SEQ ID NO: 70 - DNA, 22 bp, Artificial Sequence
  Description of Artificial Sequence Synthetic primer
actgttcttcctcagacattca

SEQ ID NO: 71 - DNA, 21 bp, Artificial Sequence
  Description of Artificial Sequence Synthetic primer
ccacagagaagttgttgaggg

SEQ ID NO: 72 - DNA, 20 bp, Artificial Sequence
  Description of Artificial Sequence Synthetic primer
gccttgtactgcagagacaa

SEQ ID NO: 73 - DNA, 18 bp, Artificial Sequence
  Description of Artificial Sequence Synthetic primer
tggtgatgggcttggtcc

SEQ ID NO: 74 - DNA, 19 bp, Artificial Sequence
  Description of Artificial Sequence Synthetic primer
tacagcggagaagggagcg

SEQ ID NO: 75 - DNA, 18 bp, Artificial Sequence
  Description of Artificial Sequence Synthetic primer
caggtaggatccagccca

SEQ ID NO: 76 - DNA, 18 bp, Artificial Sequence
  Description of Artificial Sequence Synthetic primer
atcccccaaagccaacaa

SEQ ID NO: 77 - DNA, 18 bp, Artificial Sequence
  Description of Artificial Sequence Synthetic primer
gtttgggggtgtgtggtc

SEQ ID NO: 78 - DNA, 18 bp, Artificial Sequence
  Description of Artificial Sequence Synthetic primer
ggtgcttcccattccagg

SEQ ID NO: 79 - DNA, 20 bp, Artificial Sequence
  Description of Artificial Sequence Synthetic primer
acagtcaagaagaaaacggc

SEQ ID NO: 80 - DNA, 19 bp, Artificial Sequence
  Description of Artificial Sequence Synthetic primer
agaggagctggtgttgttg

SEQ ID NO: 81 - DNA, 18 bp, Artificial Sequence
  Description of Artificial Sequence Synthetic primer
accgcttcttgtcctgct

SEQ ID NO: 82 - DNA, 20 bp, Artificial Sequence
  Description of Artificial Sequence Synthetic primer
gagcagtaaggagattcccc

SEQ ID NO: 83 - DNA, 19 bp, Artificial Sequence
  Description of Artificial Sequence Synthetic primer
aaatcggtaagaggtgggc

SEQ ID NO: 84 - DNA, 18 bp, Artificial Sequence
  Description of Artificial Sequence Synthetic primer
ccccctactgctcacctg

SEQ ID NO: 85 - DNA, 61 bp, Artificial Sequence
  Description of Artificial Sequence Synthetic primer
gtctcgtgggctcggagatgtgtataagagacagagaacaaattcctttgttatgcagaca

SEQ ID NO: 86 - DNA, 64 bp, Artificial Sequence
  Description of Artificial Sequence Synthetic primer
gtctcgtgggctcggagatgtgtataagagacagttgccaatatttaaccaattttgacctaca

SEQ ID NO: 87 - DNA, 63 bp, Artificial Sequence
  Description of Artificial Sequence Synthetic primer
gtctcgtgggctcggagatgtgtataagagacagcaagaagatgctctgagtctaatgaagtt

SEQ ID NO: 88 - DNA, 64 bp, Artificial Sequence
  Description of Artificial Sequence Synthetic primer
gtctcgtgggctcggagatgtgtataagagacagggtctataatccagatgattctttaacagg

SEQ ID NO: 89 - DNA, 56 bp, Artificial Sequence
  Description of Artificial Sequence Synthetic primer
gtctcgtgggctcggagatgtgtataagagacagctttgtccacctggaacttggt

SEQ ID NO: 90 - DNA, 64 bp, Artificial Sequence
  Description of Artificial Sequence Synthetic primer
gtctcgtgggctcggagatgtgtataagagacagctcactctaacaagcagataactttcactt

SEQ ID NO: 91 - DNA, 61 bp, Artificial Sequence
  Description of Artificial Sequence Synthetic primer
gtctcgtgggctcggagatgtgtataagagacagcagagaatgagggaggagtacattact

SEQ ID NO: 92 - DNA, 61 bp, Artificial Sequence
  Description of Artificial Sequence Synthetic primer
gtctcgtgggctcggagatgtgtataagagacagtttttgctttacgtgatgactttgttg

SEQ ID NO: 93 - DNA, 63 bp, Artificial Sequence
  Description of Artificial Sequence Synthetic primer
gtctcgtgggctcggagatgtgtataagagacaggcaaaccacaaaagtatactccatggtta

SEQ ID NO: 94 - DNA, 58 bp, Artificial Sequence
  Description of Artificial Sequence Synthetic primer
gtctcgtgggctcggagatgtgtataagagacagtccatctcttggaaactcccatct

SEQ ID NO: 95 - DNA, 62 bp, Artificial Sequence
  Description of Artificial Sequence Synthetic primer
gtctcgtgggctcggagatgtgtataagagacagaatgcaatggatgatctgggaaataaga

SEQ ID NO: 96 - DNA, 56 bp, Artificial Sequence
  Description of Artificial Sequence Synthetic primer
gtctcgtgggctcggagatgtgtataagagacagtgatgccttgacctcctgatct

SEQ ID NO: 97 - DNA, 64 bp, Artificial Sequence
  Description of Artificial Sequence Synthetic primer
gtctcgtgggctcggagatgtgtataagagacagactagagtgtctgtgtaatcaaacaagttt

SEQ ID NO: 98 - DNA, 52 bp, Artificial Sequence
  Description of Artificial Sequence Synthetic primer
gtctcgtgggctcggagatgtgtataagagacagcaggcgtcctactggcat

SEQ ID NO: 99 - DNA, 56 bp, Artificial Sequence
  Description of Artificial Sequence Synthetic primer
gtctcgtgggctcggagatgtgtataagagacagctccttcaccttgccgtaagag

SEQ ID NO: 100 - DNA, 63 bp, Artificial Sequence
  Description of Artificial Sequence Synthetic primer
gtctcgtgggctcggagatgtgtataagagacaggacatctccattcttctcttttaattgcc

SEQ ID NO: 101 - DNA, 52 bp, Artificial Sequence
  Description of Artificial Sequence Synthetic primer
gtctcgtgggctcggagatgtgtataagagacagcctagcacgtgcctacct

SEQ ID NO: 102 - DNA, 59 bp, Artificial Sequence
  Description of Artificial Sequence Synthetic primer
gtctcgtgggctcggagatgtgtataagagacagtggtgaaacctgtttgttggacata

SEQ ID NO: 103 - DNA, 58 bp, Artificial Sequence
  Description of Artificial Sequence Synthetic primer
gtctcgtgggctcggagatgtgtataagagacagcacagcaaagcagaaactcacatc

SEQ ID NO: 104 - DNA, 56 bp, Artificial Sequence
  Description of Artificial Sequence Synthetic primer
gtctcgtgggctcggagatgtgtataagagacagtgtgccagggaccttaccttat

SEQ ID NO: 105 - DNA, 55 bp, Artificial Sequence
  Description of Artificial Sequence Synthetic primer
gtctcgtgggctcggagatgtgtataagagacagatagtccaggaggcagccgaa

SEQ ID NO: 106 - DNA, 60 bp, Artificial Sequence
  Description of Artificial Sequence Synthetic primer
gtctcgtgggctcggagatgtgtataagagacagatgctgagatcagccaaattcagtta

SEQ ID NO: 107 - DNA, 57 bp, Artificial Sequence
  Description of Artificial Sequence Synthetic primer
gtctcgtgggctcggagatgtgtataagagacaggtcaagcagagaatgggtactca

SEQ ID NO: 108 - DNA, 56 bp, Artificial Sequence
  Description of Artificial Sequence Synthetic primer
gtctcgtgggctcggagatgtgtataagagacagcaggaaaatgctggctgaccta

SEQ ID NO: 109 - DNA, 58 bp, Artificial Sequence
  Description of Artificial Sequence Synthetic primer
gtctcgtgggctcggagatgtgtataagagacagtaaggcctgctgaaaatgactgaa

SEQ ID NO: 110 - DNA, 56 bp, Artificial Sequence
  Description of Artificial Sequence Synthetic primer
gtctcgtgggctcggagatgtgtataagagacagaggacttgggaggtatccacat

SEQ ID NO: 111 - DNA, 64 bp, Artificial Sequence
  Description of Artificial Sequence Synthetic primer
gtctcgtgggctcggagatgtgtataagagacagcttgctctgataggaaaatgagatctactg

SEQ ID NO: 112 - DNA, 61 bp, Artificial Sequence
  Description of Artificial Sequence Synthetic primer
gtctcgtgggctcggagatgtgtataagagacagtgtggaagatccaatccatttttgttg

SEQ ID NO: 113 - DNA, 55 bp, Artificial Sequence
  Description of Artificial Sequence Synthetic primer
gtctcgtgggctcggagatgtgtataagagacagtgaaagctgtaccatacctgt

SEQ ID NO: 114 - DNA, 58 bp, Artificial Sequence
  Description of Artificial Sequence Synthetic primer
gtctcgtgggctcggagatgtgtataagagacagaggttaatatccgcaaatgacttg

SEQ ID NO: 115 - DNA, 55 bp, Artificial Sequence
  Description of Artificial Sequence Synthetic primer
gtctcgtgggctcggagatgtgtataagagacagagatgatccgacaagtgagag

SEQ ID NO: 116 - DNA, 52 bp, Artificial Sequence
  Description of Artificial Sequence Synthetic primer
gtctcgtgggctcggagatgtgtataagagacagtacgtctcctccgaccac

SEQ ID NO: 117 - DNA, 52 bp, Artificial Sequence
  Description of Artificial Sequence Synthetic primer
gtctcgtgggctcggagatgtgtataagagacagcgcagcctgtacccagtg

SEQ ID NO: 118 - DNA, 56 bp, Artificial Sequence
  Description of Artificial Sequence Synthetic primer
gtctcgtgggctcggagatgtgtataagagacaggcggaagatgaagatttcggat

SEQ ID NO: 119 - DNA, 59 bp, Artificial Sequence
  Description of Artificial Sequence Synthetic primer
gtctcgtgggctcggagatgtgtataagagacaggttagctcatttttgttaatggtgg

SEQ ID NO: 120 - DNA, 60 bp, Artificial Sequence
  Description of Artificial Sequence Synthetic primer
gtctcgtgggctcggagatgtgtataagagacagttaaacttttcttttagttgtgctga

SEQ ID NO: 121 - DNA, 61 bp, Artificial Sequence
  Description of Artificial Sequence Synthetic primer
gtctcgtgggctcggagatgtgtataagagacaggtatgcaacatttctaaagttacctac

SEQ ID NO: 122 - DNA, 54 bp, Artificial Sequence
  Description of Artificial Sequence Synthetic primer
gtctcgtgggctcggagatgtgtataagagacagaagaccataacccaccacag

SEQ ID NO: 123 - DNA, 54 bp, Artificial Sequence
  Description of Artificial Sequence Synthetic primer
gtctcgtgggctcggagatgtgtataagagacagctggaaagggacgaactggt

SEQ ID NO: 124 - DNA, 54 bp, Artificial Sequence
  Description of Artificial Sequence Synthetic primer
gtctcgtgggctcggagatgtgtataagagacagcgacccagttaccatagcaa

SEQ ID NO: 125 - DNA, 60 bp, Artificial Sequence
  Description of Artificial Sequence Synthetic primer
gtctcgtgggctcggagatgtgtataagagacagagaaaatggaagtctatgtgatcaag

SEQ ID NO: 126 - DNA, 63 bp, Artificial Sequence
  Description of Artificial Sequence Synthetic primer
gtctcgtgggctcggagatgtgtataagagacagcattttaaattttctttctctaggtgaag

SEQ ID NO: 127 - DNA, 52 bp, Artificial Sequence
  Description of Artificial Sequence Synthetic primer
gtctcgtgggctcggagatgtgtataagagacagggtcttggccgaggtctc

SEQ ID NO: 128 - DNA, 52 bp, Artificial Sequence
  Description of Artificial Sequence Synthetic primer
gtctcgtgggctcggagatgtgtataagagacagcagcaccatgggcacgtc

SEQ ID NO: 129 - DNA, 53 bp, Artificial Sequence
  Description of Artificial Sequence Synthetic primer
gtctcgtgggctcggagatgtgtataagagacaggagaggtggaaagcgagag

SEQ ID NO: 130 - DNA, 52 bp, Artificial Sequence
  Description of Artificial Sequence Synthetic primer
gtctcgtgggctcggagatgtgtataagagacagcactcttgcccacaccgc

SEQ ID NO: 131 - DNA, 62 bp, Artificial Sequence
  Description of Artificial Sequence Synthetic primer
gtctcgtgggctcggagatgtgtataagagacagctgtatttatttcagtgttacttacctg

SEQ ID NO: 132 - DNA, 64 bp, Artificial Sequence
  Description of Artificial Sequence Synthetic primer
gtctcgtgggctcggagatgtgtataagagacagttatattcaatttaaacccacctataatgg

SEQ ID NO: 133 - DNA, 56 bp, Artificial Sequence
  Description of Artificial Sequence Synthetic primer
gtctcgtgggctcggagatgtgtataagagacaggtatcaaagaatggtcctgcac

SEQ ID NO: 134 - DNA, 57 bp, Artificial Sequence
  Description of Artificial Sequence Synthetic primer
gtctcgtgggctcggagatgtgtataagagacagacacaacacaaaatagccgtata

SEQ ID NO: 135 - DNA, 59 bp, Artificial Sequence
  Description of Artificial Sequence Synthetic primer
gtctcgtgggctcggagatgtgtataagagacagtctattctttcctttgtagtgtcca

SEQ ID NO: 136 - DNA, 61 bp, Artificial Sequence
  Description of Artificial Sequence Synthetic primer
gtctcgtgggctcggagatgtgtataagagacagtgatttctgtttttacctcctaaagaa

SEQ ID NO: 137 - DNA, 64 bp, Artificial Sequence
  Description of Artificial Sequence Synthetic primer
gtctcgtgggctcggagatgtgtataagagacagatctttaaagagaaatttgctaaagctgtg

SEQ ID NO: 138 - DNA, 57 bp, Artificial Sequence
  Description of Artificial Sequence Synthetic primer
gtctcgtgggctcggagatgtgtataagagacagacgtgttttgatcaaagaagagg

SEQ ID NO: 139 - DNA, 52 bp, Artificial Sequence
  Description of Artificial Sequence Synthetic primer
gtctcgtgggctcggagatgtgtataagagacagagcctcacgttggtccac

SEQ ID NO: 140 - DNA, 52 bp, Artificial Sequence
  Description of Artificial Sequence Synthetic primer
gtctcgtgggctcggagatgtgtataagagacagggatggctaggcgaggag

SEQ ID NO: 141 - DNA, 52 bp, Artificial Sequence
  Description of Artificial Sequence Synthetic primer
gtctcgtgggctcggagatgtgtataagagacagagggactaggcgtgggat

SEQ ID NO: 142 - DNA, 52 bp, Artificial Sequence
  Description of Artificial Sequence Synthetic primer
gtctcgtgggctcggagatgtgtataagagacagccaagccctagggtggtg

SEQ ID NO: 143 - DNA, 55 bp, Artificial Sequence
  Description of Artificial Sequence Synthetic primer
gtctcgtgggctcggagatgtgtataagagacagacgctcttctcactcatatcc

SEQ ID NO: 144 - DNA, 59 bp, Artificial Sequence
  Description of Artificial Sequence Synthetic primer
gtctcgtgggctcggagatgtgtataagagacagaagaaatcttagacgtaagcccctc

SEQ ID NO: 145 - DNA, 52 bp, Artificial Sequence
  Description of Artificial Sequence Synthetic primer
gtctcgtgggctcggagatgtgtataagagacagtcccataccctctcagcg

SEQ ID NO: 146 - DNA, 54 bp, Artificial Sequence
  Description of Artificial Sequence Synthetic primer
gtctcgtgggctcggagatgtgtataagagacagggatgagctacctggaggat

SEQ ID NO: 147 - DNA, 58 bp, Artificial Sequence
  Description of Artificial Sequence Synthetic primer
gtctcgtgggctcggagatgtgtataagagacagattttgagtgttagactggaaact

SEQ ID NO: 148 - DNA, 53 bp, Artificial Sequence
  Description of Artificial Sequence Synthetic primer
gtctcgtgggctcggagatgtgtataagagacaggggcagtgctaggaaagag

SEQ ID NO: 149 - DNA, 53 bp, Artificial Sequence
  Description of Artificial Sequence Synthetic primer
gtctcgtgggctcggagatgtgtataagagacagtgcttacctcgcttagtgc

SEQ ID NO: 150 - DNA, 52 bp, Artificial Sequence
  Description of Artificial Sequence Synthetic primer
gtctcgtgggctcggagatgtgtataagagacaggccggggatgtgatgaga

SEQ ID NO: 151 - DNA, 52 bp, Artificial Sequence
  Description of Artificial Sequence Synthetic primer
gtctcgtgggctcggagatgtgtataagagacagcaggggtcagaggcaagc

SEQ ID NO: 152 - DNA, 52 bp, Artificial Sequence
  Description of Artificial Sequence Synthetic primer
gtctcgtgggctcggagatgtgtataagagacagagggccactgacaaccac